SEQUENCE LISTING (66 sequences)

SEQ ID NO: 1 - protein, 10 aa, synthetic construct
GDTFGITVIG

SEQ ID NO: 2 - protein, 10 aa, synthetic construct
GFTLSTLDMR

SEQ ID NO: 3 - protein, 10 aa, synthetic construct
GFDFSRYDMG

SEQ ID NO: 4 - protein, 10 aa, synthetic construct
GSXXRVNAMG

SEQ ID NO: 5 - protein, 10 aa, synthetic construct
GNTFSINSMA

SEQ ID NO: 6 - protein, 10 aa, synthetic construct
ARIYNTGTVK

SEQ ID NO: 7 - protein, 10 aa, synthetic construct
STVSPDGNTY

SEQ ID NO: 8 - protein, 11 aa, synthetic construct
SGIKSGGGPTV

SEQ ID NO: 9 - protein, 10 aa, synthetic construct
AAIDRSGATV

SEQ ID NO: 10 - protein, 15 aa, synthetic construct
AELHKTDRPGSVAAN

SEQ ID NO: 11 - protein, 13 aa, synthetic construct
AMGEADLTHYDLW

SEQ ID NO: 12 - protein, 15 aa, synthetic construct
ADISIAAHEGGLPYW

SEQ ID NO: 13 - protein, 14 aa, synthetic construct
DPDDHSGSYWGDYW

SEQ ID NO: 14 - protein, 7 aa, synthetic construct
SGVLGSW

SEQ ID NO: 15 - protein, 11 aa, synthetic construct
ATGLWRDHEVW

SEQ ID NO: 16 - protein, 25 aa, synthetic construct
QVQLVESGGGLVQAGESLKLSCKAS

SEQ ID NO: 17 - protein, 25 aa, synthetic construct
QVQLVQSGGGLVQPGGSLRLSCAAS

SEQ ID NO: 18 - protein, 25 aa, synthetic construct
DVQLVESGGGMVQPGGSLRLSCVTS

SEQ ID NO: 19 - protein, 25 aa, synthetic construct
QVQLVESGGGLAQAGGSLQLSCAAS

SEQ ID NO: 20 - protein, 25 aa, synthetic construct
QVQLVESGGGLVQAGESLNLSCTAS

SEQ ID NO: 21 - protein, 14 aa, synthetic construct
WHRQRAGQQQRELV

SEQ ID NO: 22 - protein, 13 aa, synthetic construct
WFRQAPGKGFEWV

SEQ ID NO: 23 - protein, 13 aa, synthetic construct
WVRQAPGKGPEWV

SEQ ID NO: 24 - protein, 13 aa, synthetic construct
WYRQTPGKERELV

SEQ ID NO: 25 - protein, 13 aa, synthetic construct
WYRQAPGKQRELV

SEQ ID NO: 26 - protein, 38 aa, synthetic construct
YADSVKGRFTLSSDSANDVVYLEMTDLKPEDTAVYYCH

SEQ ID NO: 27 - protein, 38 aa, synthetic construct
YSDSAKGRFAISRDVAKNTVNLQMSSLKPEDTAVYYCN

SEQ ID NO: 28 - protein, 38 aa, synthetic construct
YLDSVKGRFTVSRDNAKNTLYLQMNNLKPEDTARYYCA

SEQ ID NO: 29 - protein, 38 aa, synthetic construct
YSESVRGRFTISKNDXKXIVWLQMNNLTTEDTAVYYCR

SEQ ID NO: 30 - protein, 38 aa, synthetic construct
YADSVKGRFTIATDNSRDTMYLQMTNLKSADTATYFCY

SEQ ID NO: 31 - protein, 10 aa, synthetic construct
GPGTQVTVSS

SEQ ID NO: 32 - protein, 10 aa, synthetic construct
GKGTPVTVSS

SEQ ID NO: 33 - protein, 10 aa, synthetic construct
GQGTLVTVSS

SEQ ID NO: 34 - protein, 10 aa, synthetic construct
GLGTQVTVSS

SEQ ID NO: 35 - protein, 10 aa, synthetic construct
GQGTLVTVSS

SEQ ID NO: 36 - protein, 120 aa, synthetic construct
QVQLVESGGGLVQAGESLKLSCKASGDTFGITVIGWHRQRAGQQQRELVARIYNTGTVKYADSVKGRFTLSSDSANDVVYLEMTDLKPEDTAVYYCHAMGEADLTHYDLWGPGTQVTVSS

SEQ ID NO: 37 - protein, 121 aa, synthetic construct
QVQLVQSGGGLVQPGGSLRLSCAASGFTLSTLDMRWFRQAPGKGFEWVSTVSPDGNTYYSDSAKGRFAISRDVAKNTVNLQMSSLKPEDTAVYYCNADISIAAHEGGLPYWGKGTPVTVSS

SEQ ID NO: 38 - protein, 122 aa, synthetic construct
DVQLVESGGGMVQPGGSLRLSCVTSGFDFSRYDMGWVRQAPGKGPEWVSGIKSGGGPTVYLDSVKGRFTVSRDNAKNTLYLQMNNLKPEDTARYYCARDPDDHSGSYWGDYWGQGTLVTVSS

SEQ ID NO: 39 - protein, 113 aa, synthetic construct
QVQLVESGGGLAQAGGSLQLSCAASGSTSRVNAMGWYRQTPGKERELVAAIDRSGATVYSESVRGRFTISKNDAKNIVWLQMNNLTTEDTAVYYCRSGVLGSWGLGTQVTVSS

SEQ ID NO: 40 - protein, 122 aa, synthetic construct
QVQLVESGGGLVQAGESLNLSCTASGNTFSINSMAWYRQAPGKQRELVAELHKTDRPGSVAANYADSVKGRFTIATDNSRDTMYLQMTNLKSADTATYFCYATGLWRDHEVWGQGTLVTVSS

SEQ ID NO: 41 - protein, 113 aa, synthetic construct
QVQLVESGGGLAQAGGSLQLSCAASGSTSRVNAMGWYRQTPGKERELVAAIDRSGATVYSESVRGRFTISKNDAKDIVWLQMNNLTTEDTAVYYCRSGVLGSWGLGTQVTVSS

SEQ ID NO: 42 - protein, 113 aa, synthetic construct
QVQLVESGGGLAQAGGSLQLSCAASGSTSRVNAMGWYRQTPGKERELVAAIDRSGATVYSESVRGRFTISKNDYKDIVWLQMNNLTTEDTAVYYCRSGVLGSWGLGTQVTVSS

SEQ ID NO: 43 - protein, 113 aa, synthetic construct
QVQLVESGGGLAQAGGSLQLSCAASGSTYRVNAMGWYRQTPGKERELVAAIDRSGATVYSESVRGRFTISKNDAKDIVWLQMNNLTTEDTAVYYCRSGVLGSWGLGTQVTVSS

SEQ ID NO: 44 - protein, 113 aa, synthetic construct
QVQLVESGGGLAQAGGSLQLSCAASGSTYRVNAMGWYRQTPGKERELVAAIDRSGATVYSESVRGRFTISKNDYKDIVWLQMNNLTTEDTAVYYCRSGVLGSWGLGTQVTVSS

SEQ ID NO: 45 - protein, 5 aa, synthetic construct
GSGGS

SEQ ID NO: 46 - protein, 4 aa, synthetic construct
GGGS

SEQ ID NO: 47 - protein, 5 aa, synthetic construct
GGGGS

SEQ ID NO: 48 - protein, 9 aa, synthetic construct
GGGGSGGGS

SEQ ID NO: 49 - protein, 15 aa, synthetic construct
GGGGSGGGGSGGGGS

SEQ ID NO: 50 - protein, 5 aa, synthetic construct
GPGGP

SEQ ID NO: 51 - protein, 36 aa, synthetic construct
AALVGPGGQGGGGSGGGGSGGGGSGGGGSGGGGSMA

SEQ ID NO: 52 - protein, 15 aa, synthetic construct
EPKSSDKTHTSPPSP

SEQ ID NO: 53 - protein, 12 aa, synthetic construct
GPGGQGTGPGGS

SEQ ID NO: 54 - protein, 22 aa, synthetic construct
MKYLLPTAAAGLLLLAAQPAMA

SEQ ID NO: 55 - protein, 11 aa, synthetic construct
GPGGQHHHHHH

SEQ ID NO: 56 - protein, 12 aa, synthetic construct
GAYPYDVPDYAS

SEQ ID NO: 57 - DNA, 498 bp, synthetic construct
  source 1..498: mol_type = other DNA
atgaaatacctattgcctacggcggccgctggattgttattactcgcggcccagccggccatggctcaggtgcagctcgtggagtctgggggaggcttggtgcaggctggagagtctctgaaactctcctgtaaggcctctggagataccttcggtatcaccgtcatcggctggcaccgccaacgtgcaggtcaacagcagcgcgaattggtcgcgcgcatttataatactgggactgtaaaatacgccgactccgtgaagggccggttcaccctctccagtgacagcgccaacgacgtggtgtatctggagatgactgacttgaaaccggaagacacggccgtctattactgccacgcgatgggcgaagctgacctcactcactatgacctctggggcccggggacccaggtcaccgtctccagcggcccgggaggccaacaccatcaccaccatcatggcgcatatccgtatgatgtgccggactatgcttcttag

SEQ ID NO: 58 - protein, 165 aa, synthetic construct
MKYLLPTAAAGLLLLAAQPAMAQVQLVESGGGLVQAGESLKLSCKASGDTFGITVIGWHRQRAGQQQRELVARIYNTGTVKYADSVKGRFTLSSDSANDVVYLEMTDLKPEDTAVYYCHAMGEADLTHYDLWGPGTQVTVSSGPGGQHHHHHHGAYPYDVPDYAS

SEQ ID NO: 59 - DNA, 501 bp, synthetic construct
  source 1..501: mol_type = other DNA
atgaaatacctattgcctacggcggccgctggattgttattactcgcggcccagccggccatggctcaggtgcagctcgtgcagtctgggggaggcttggtgcaacctgggggttctctgagactctcctgtgcagcctctggcttcaccctcagtacgttggatatgaggtggttccgccaggctccaggaaaggggttcgagtgggtctcaactgttagtcctgatggtaacacgtactactcagactccgcgaagggccgattcgccatctccagagacgtcgccaagaacacggtgaatctgcaaatgagcagcctgaaacctgaagacacggccgtctattattgtaatgcagatatttcaatcgcggcgcacgagggcggactgccctactggggcaaagggaccccggtcaccgtctccagcggcccgggaggccaacaccatcaccaccatcatggcgcatatccgtatgatgtgccggactatgcttcttag

SEQ ID NO: 60 - protein, 166 aa, synthetic construct
MKYLLPTAAAGLLLLAAQPAMAQVQLVQSGGGLVQPGGSLRLSCAASGFTLSTLDMRWFRQAPGKGFEWVSTVSPDGNTYYSDSAKGRFAISRDVAKNTVNLQMSSLKPEDTAVYYCNADISIAAHEGGLPYWGKGTPVTVSSGPGGQHHHHHHGAYPYDVPDYAS

SEQ ID NO: 61 - DNA, 504 bp, synthetic construct
  source 1..504: mol_type = other DNA
atgaaatacctattgcctacggcggccgctggattgttattactcgcggcccagccggccatggctgatgttcagctcgtggagtctgggggaggcatggtgcagcctggggggtctctgagactctcctgtgtaacctctggattcgacttcagccgctatgatatgggctgggtccgccaggctccagggaaggggcccgagtgggtctcaggaattaaaagtggtggcggtcccacagtctacctagactccgtgaagggccgattcaccgtctccagagataacgccaagaatactttatatctccaaatgaacaacctgaaacctgaggacacggcccggtattactgtgcgagagatcccgacgaccacagtggtagttactggggcgactactggggccaggggaccctggtcaccgtctccagcggcccgggaggccaacaccatcaccaccatcatggcgcatatccgtatgatgtgccggactatgcttcttag

SEQ ID NO: 62 - protein, 167 aa, synthetic construct
MKYLLPTAAAGLLLLAAQPAMADVQLVESGGGMVQPGGSLRLSCVTSGFDFSRYDMGWVRQAPGKGPEWVSGIKSGGGPTVYLDSVKGRFTVSRDNAKNTLYLQMNNLKPEDTARYYCARDPDDHSGSYWGDYWGQGTLVTVSSGPGGQHHHHHHGAYPYDVPDYAS

SEQ ID NO: 63 - DNA, 477 bp, synthetic construct
  source 1..477: mol_type = other DNA
atgaaatacctattgcctacggcggccgctggattgttattactcgcggcccagccggccatggctcaggtgcagctcgtggagtccgggggaggcttggcgcaggctgggggatctctgcaactctcgtgcgcagcttccggtagtacctccagagtcaatgccatgggttggtaccgccagactccggggaaagagcgcgagttggtcgcggcgattgaccgctccggagccacggtctactcggagtccgtgaggggacgattcaccatctccaaaaacgatgccaaaaacatcgtgtggctgcaaatgaacaacctgacgactgaagatacggccgtctattactgtcgcagtggggtccttggttcctggggcctggggacccaggtcaccgtctccagcggcccgggaggccaacaccatcaccaccatcatggcgcatatccgtatgatgtgccggactatgcttcttag

SEQ ID NO: 64 - protein, 158 aa, synthetic construct
MKYLLPTAAAGLLLLAAQPAMAQVQLVESGGGLAQAGGSLQLSCAASGSTSRVNAMGWYRQTPGKERELVAAIDRSGATVYSESVRGRFTISKNDAKNIVWLQMNNLTTEDTAVYYCRSGVLGSWGLGTQVTVSSGPGGQHHHHHHGAYPYDVPDYAS

SEQ ID NO: 65 - DNA, 504 bp, synthetic construct
  source 1..504: mol_type = other DNA
atgaaatacctattgcctacggcggccgctggattgttattactcgcggcccagccggccatggctcaggtgcagctcgtggagtctgggggaggcttggtgcaggctggagaatctctgaatctctcctgcacagcctccgggaacaccttcagtatcaattccatggcctggtaccgccaggctccaggaaagcagcgcgagttggtcgcggaacttcataaaactgatcggccaggtagtgtcgccgcgaactatgcagactccgtgaagggccgattcaccatcgccaccgacaactccagagacacgatgtatctccaaatgaccaacctaaaatctgcggatactgccacctatttctgttatgcgacggggctctggagggatcatgaagtttggggccagggcaccctggtcaccgtctccagcggcccgggaggccaacaccatcaccaccatcatggcgcatatccgtatgatgtgccggactatgcttcttag

SEQ ID NO: 66 - protein, 167 aa, synthetic construct
MKYLLPTAAAGLLLLAAQPAMAQVQLVESGGGLVQAGESLNLSCTASGNTFSINSMAWYRQAPGKQRELVAELHKTDRPGSVAANYADSVKGRFTIATDNSRDTMYLQMTNLKSADTATYFCYATGLWRDHEVWGQGTLVTVSSGPGGQHHHHHHGAYPYDVPDYAS

(19) United States
(12) Patent Application Publication
(10) Pub. No.: US 2025/0243296 A1
(43) Pub. Date: Jul. 31, 2025

(54) SINGLE-DOMAIN ANTIBODIES AND VARIANTS THEREOF AGAINST OPIOIDS

(71) Applicants: Wisconsin Alumni Research Foundation, Madison, WI (US); University of Minnesota Technology Commercialization, Minneapolis, MN (US); University of Washington CoMotion, Seattle, WA (US)

(72) Inventors: Aaron LeBeau, Middleton, WI (US); Joseph Gallant, Middleton, WI (US); Marco Pravetoni, Seattle, WA (US)

(73) Assignee: Wisconsin Alumni Research Foundation, Madison, WI (US)

(21) Appl. No.: 19/041,689

(22) Filed: Jan. 30, 2025

Related U.S. Application Data
(60) Provisional application No. 63/627,144, filed on Jan. 31, 2024.

(51) Int. Cl. C07K 16/44; A61K 39/00

(52) U.S. Cl. CPC C07K 16/44; A61K 2039/505; C07K 2317/22; C07K 2317/33; C07K 2317/569; C07K 2317/92

(57) ABSTRACT

The present application provides constructs comprising a single-domain antibody (sdAb) moiety that specifically recognizes opioids such as fentanyl and carfentanil. Also provided are methods of making and using these constructs.

### CROSS-REFERENCE TO RELATED APPLICATIONS

Priority is hereby claimed to U.S. Provisional Application 63/627,144, filed Jan. 31, 2024, which is incorporated herein by reference in its entirety.

### SEQUENCE LISTING

The instant application contains a Sequence Listing which has been submitted in XML format and is hereby incorporated by reference in its entirety. The XML copy, created on Jan. 21, 2025, is named USPTO-09824581-P230209US02-SEQ_LIST.XML and is 62,733 bytes in size.

### FIELD OF THE INVENTION

The present invention relates to constructs comprising single-domain antibody (sdAb) moieties that specifically recognize opioids such as fentanyl and carfentanil, and methods of making and using thereof.

### BACKGROUND

It has been estimated that opioid use disorder (OUD) affects over 2.7 million people in the United States alone (Centers for Disease Control and Prevention, 2022). Widespread access to opioids has concurrently resulted in a dramatic increase of fatal overdoses in recent years¹.

In 2022, there were more than 79,000 reported opioid related overdose deaths in the United States. Fatal overdose involving opioids is now the leading cause of death for individuals under the age of 50². The recent surge in fatal overdoses can be attributed to the influx of illegally made and imported synthetic opioids. Nearly two-thirds of the opioid-related overdose deaths last year were due to a single synthetic opioid, fentanyl³⁻⁴. Fentanyl belongs to a class of synthetic piperidine-based opioids that act as agonists for the p-opioid receptors (MORs) in the brain⁵. When compared to opiates originating from natural products, fentanyl is 50 to 100 times more potent than heroin and morphine^{5,6}. Commonly used for pain management and as a sedative, fentanyl is fast acting and can diffuse across the blood-brain barrier⁶. Due to its enhanced potency and extended serum half-life (t_{1/2}-8 h) compared to traditional opioids, fentanyl is frequently misused resulting in severe respiratory depression that ultimately leads to overdose and death^{5,7}. Fentanyl is also commonly used to lace counterfeit pills and other drugs resulting in an increased number of people unknowingly overdosing on fentanyl^{8,9}.

The most commonly used therapeutic to reverse opioid-related overdose is the MORs antagonist naloxone. While naloxone is generally effective at reversing overdose, the duration of its action is short and it is rapidly cleared from the brain¹⁰. The transient effect and short half-life of naloxone (30-90 min) can result in renarcotization when used to treat overdose caused by longer-lived, more potent synthetic MORs agonists¹⁰⁻¹². This has led to the investigation of immunotherapies for overdose that alter the pharmacokinetics of the target opioid by sequestering it in the serum and preventing its distribution to the MORs in the brain¹³⁻¹⁴. Anti-opioid vaccines and monoclonal antibodies (mAbs) can offer effective protection for weeks and months unlike small-molecule antagonists¹⁵. Anti-opioid vaccines that consist of an opioid-based hapten conjugated to an immunogenic carrier protein act by generating a polyclonal antibody response to clear the target opioid¹⁶⁻¹⁸. Anti-opioid vaccines have demonstrated efficacy at reducing opioid biodistribution, respiratory depression, and death in preclinical rodent and non-human primate studies¹⁹⁻²¹. A multi-center first-in-human clinical trial for a vaccine to treat OUD is currently ongoing (NCT04458545). A successful vaccine is dependent on the generation of high levels of polyclonal antibodies against the target opioid²². This may require multiple immunizations to achieve high antibody concentrations in a subset of patients. To circumvent the variability of the immune response, mAbs that recognize opioids including fentanyl have been developed²³⁻²⁵. The advantages of mAbs is that they allow for immediate protection against the target opioid and their serum concentrations can be adjusted to counteract the presence of long-lived synthetic opioids²⁶. Recently, the human IgG1 mAb CSX-1004 received fast track designation for the prevention of overdose from fentanyl and other synthetic opioids (NCT06005402)²⁴.

Monoclonal antibodies are a promising class of therapeutics to counteract the effects of opioids. All of the anti-opioid and other commonly abused small-molecule drug mAbs were initially identified using traditional hybridoma technology from rodents or humanized transgenic rodent models. The anti-opioid mAbs in the literature are all canonical heavy and light chain antibodies. Single-domain antibodies represent an unexplored technology for the development of next-generation mAbs for opioids. Variable-heavy-heavy domains (VHHs) or nanobodies are single-domain heavy-chain only fragments found in members of the camelidae family (camels, llamas, alpacas, etc.)²⁷. VHHs have three CDRs for target engagement as opposed to the six CDRs (3 heavy, 3 light) found in human antibodies. However, the CDR3 of camelids can be over 20 amino acid residues long²⁸. This provides them with a unique architecture and binding interface that allows them to recognize epitopes inaccessible to canonical human and mouse antibodies^{29,30}. VHHs are easy to express in large quantities because of their high solubility and they require little to no humanization due to their high homology with human heavy chains (>95%) which makes VHHs ideal for translation^{28,31}.

There is a critical unmet need for effective antibody-based therapies that can target opioids such as fentanyl and carfentanil for mitigating the effects of the opioids, such as in cases of overdose.

### SUMMARY OF THE INVENTION

One aspect of the invention is directed isolated anti-opioid constructs comprising single-domain antibody (sdAb) moieties specifically recognizing an opioid selected from the group consisting of fentanyl and carfentanil.

In some versions, the sdAb moiety comprises a CDR1, a CDR2, and a CDR3.

In some versions, the CDR1 comprises an amino acid sequence of GDTFGITVIG (SEQ ID NO: 1), GFTLSTLDMR (SEQ ID NO: 2), GFDFSRYDMG (SEQ ID NO: 3), GSX¹X²RVNAMG (SEQ ID NO: 4) wherein X¹ is T or D and X² is S or Y, GNTFSINSMA (SEQ ID NO: 5), or a variant of any of the foregoing comprising up to about 3 (such as about any of 1, 2, or 3) amino acid substitutions.

In some versions, the CDR2 comprises an amino acid sequence of ARIYNTGTVK (SEQ ID NO: 6), STVSPDGNTY (SEQ ID NO: 7), SGIKSGGGPTV (SEQ ID NO: 8), AAIDRSGATV (SEQ ID NO: 9), AELHKTDRPGSVAAN (SEQ ID NO: 10), or a variant of any of the foregoing comprising up to about 3 (such as about any of 1, 2, or 3) amino acid substitutions.

In some versions, the CDR3 comprises an amino acid sequence of AMGEADLTHYDLW (SEQ ID NO: 11), ADISIAAHEGGLPYW (SEQ ID NO: 12), DPDDHSGSYWGDYW (SEQ ID NO: 13), SGVLGSW (SEQ ID NO: 14), ATGLWRDHEVW (SEQ ID NO: 15), or a variant of any of the foregoing comprising up to about 3 (such as about any of 1, 2, or 3) amino acid substitutions.

In some versions, the sdAb moiety comprises an FR1, an FR2, an FR3, and an FR4.

In some versions, the FR1 comprises an amino acid sequence of QVQLVESGGGLVQAGESLKLSCKAS (SEQ ID NO: 16), QVQLVQSGGGLVQPGGSLRLSCAAS (SEQ ID NO: 17), DVQLVESGGGMVQPGGSLRLSCVTS (SEQ ID NO: 18), QVQLVESGGGLAQAGGSLQLSCAAS (SEQ ID NO: 19), QVQLVESGGGLVQAGESLNLSCTAS (SEQ ID NO: 20), or a variant of any of the foregoing comprising up to about 3 (such as about any of 1, 2, or 3) amino acid substitutions.

In some versions, the FR2 comprises an amino acid sequence of WHRQRAGQQQRELV (SEQ ID NO: 21), WFRQAPGKGFEWV (SEQ ID NO: 22), WVRQAPGKGPEWV (SEQ ID NO: 23), WYRQTPGKERELV (SEQ ID NO: 24), WYRQAPGKQRELV (SEQ ID NO: 25), or a variant of any of the foregoing comprising up to about 3 (such as about any of 1, 2, or 3) amino acid substitutions.

In some versions, the FR3 comprises an amino acid sequence of YADSVKGRFTLSSDSANDVVYLEMTDLKPEDTAVYYCH (SEQ ID NO: 26), YSDSAKGRFAISRDVAKNTVNLQMSSLKPEDTAVYYCN (SEQ ID NO: 27), YLDSVKGRFTVSRDNAKNTLYLQMNNLKPEDTARYYCA (SEQ ID NO: 28), YSESVRGRFTISKNDX³KX⁴IVWLQMNNLTTEDTAVYYCR (SEQ ID NO: 29) wherein X³ is A, Y, or W and X⁴ is N, D, H, or Y, YADSVKGRFTIATDNSRDTMYLQMTNLKSADTATYFCY (SEQ ID NO: 30), or a variant of any of the foregoing comprising up to about 3 (such as about any of 1, 2, or 3) amino acid substitutions. In some versions, the FR3 comprises an amino acid sequence of YSESVRGRFTISKNDX³KX⁴IVWLQMNNLTTEDTAVYYCR (SEQ ID NO: 29) wherein X³ is A, Y, or W and X⁴ is D or a variant thereof comprising up to 3 amino acid substitutions with the proviso that the D at X⁴ is not substituted.

In some versions, the FR4 comprises an amino acid sequence of GPGTQVTVSS (SEQ ID NO: 31), GKGTPVTVSS (SEQ ID NO: 32), GQGTLVTVSS (SEQ ID NO: 33), GLGTQVTVSS (SEQ ID NO: 34), GQGTLVTVSS (SEQ ID NO: 35), or a variant of any of the foregoing comprising up to about 3 (such as about any of 1, 2, or 3) amino acid substitutions.

Another aspect of the invention is directed to pharmaceutical compositions. In some versions, the pharmaceutical compositions comprise an isolated anti-opioid construct of the invention a nucleic acid configured to express the isolated anti-opioid construct.

Another aspect of the invention is directed to methods of using the isolated anti-opioid constructs of the invention, the nucleic acids configured to express the isolated anti-opioid constructs of the invention, and the pharmaceutical compositions of the invention. The methods can comprise administering the isolated anti-opioid constructs, nucleic acids, or pharmaceutical compositions to an individual to thereby bind the anti-opioid construct to an opioid present within a body of the individual. The opioid can be selected from the group consisting of fentanyl and carfentanil.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. (FIG. 1A) Structures of Fentanyl (CAS: 437-38-7) and Carfentanil (CAS: 59708-52-0) with structural differences highlighted in red. (FIG. 1B) Camelid phage display library used for biopanning campaign and the resulting phage titers following each round. (FIG. 1C) Dilution ELISA of the 5 lead anti-fentanyl camelids against an immobilized F3 (fentanyl) hapten showing saturable binding profiles. (FIG. 1D) The highest concentration from the dilution ELISA in (FIG. 1C) was evaluated in a single concentration ELISA against the immobilized F11 (carfentanil) hapten.

FIGS. 2A-2F. (FIG. 2A) Overall structure of the domain-swapped JGFN4 dimer bound to fentanyl. (FIG. 2B) The CDR 1-3 loops of JGFN4 in the domain-swapped ‘trans’ configuration. (FIG. 2C) 2Fo-Fc electron density contoured at 1.0 sigma. (FIG. 2D) A close-up view of the fentanyl-binding pocket, with the side chains of key amino acid residues shown. Yellow dashed lines indicate hydrogen bonds, with distances shown in angstroms. (FIG. 2E) Superposition between a fentanyl-free JGFN4 monomer (beige) and a fentanyl-bound JGFN4 protomer from with the domain-swapped homodimer (cyan), highlighting distinct configurations of the C-terminal beta-strand. (FIG. 2F) A close-up view of the superposition in E, showing different conformations of CDR3 and the neighboring CDR1 including Asn32.

FIGS. 3A and 3B. (FIG. 3A) A comparison between the fentanyl-bound JGFN4 N76D monomer (orange) and a protomer of fentanyl-bound JGFN4 N76D within the domain-swapped homodimer (cyan). Note the distinct positioning of Asn32. (FIG. 3B) A comparison between the fentanyl-bound JGFN4 WT (magenta) and N76D (cyan), both from domain-swapped homodimers. Note that a hydrogen bond with Asp76 brings Thr28 closer to fentanyl.

### DETAILED DESCRIPTION OF THE INVENTION

The present invention provides single-domain antibodies (sdAbs) specifically recognizing opioids such as fentanyl and carfentanil (hereinafter also referred to as “anti-opioid sdAbs”) and their antibody variants.

Single-chain antibodies (sdAbs) are different from conventional 4-chain antibodies by having a single monomeric antibody variable domain, such as heavy chain variable domain (VHH, also abbreviated in the art as VHH), which can exhibit high affinity to an antigen without the aid of a light chain. Camelid VHHs are known as some of the smallest functional antigen-binding fragments with molecular weights of approximately 15 kD.

Accordingly, one aspect of the present application provides isolated anti-opioid constructs comprising sdAb moieties specifically recognizing opioids such as fentanyl and carfentanil. The isolated anti-opioid constructs can be, for example, an anti-opioid sdAb, a polypeptide comprising multiple anti-opioid sdAbs fused together, and other constructs. The anti-opioid constructs can be monospecific or multispecific, monovalent, or multivalent.

Also provided are compositions (such as pharmaceutical compositions) and methods of using the anti-opioid constructs for, e.g., treating opioid (fentanyl or carfentanil) overdoses and other uses.

### Anti-Opioid Constructs

### Anti-Opioid Single-Domain Antibody Moiety

The isolated anti-opioid constructs described herein can comprise a single-domain antibody (sdAb) moiety that specifically recognizes an opioid such as fentanyl or carfentanil (“anti-opioid sdAb”). In some embodiments, the isolated anti-opioid construct is an anti-opioid sdAb.

### Single-Domain Antibodies

Exemplary sdAbs include, but are not limited to, heavy chain variable domains from heavy-chain only antibodies (e.g., VHH (variable domain of the heavy chain of the heavy chain antibody) in Camelidae or V_{NAR} (Variable domain of the shark New Antigen Receptor) in cartilaginous fish), binding molecules naturally devoid of light chains, single domains (such as V_{H} or V_{L}) derived from conventional 4-chain antibodies, humanized heavy-chain only antibodies, human single-domain antibodies produced by transgenic mice or rats expressing human heavy chain segments, and engineered domains and single domain scaffolds other than those derived from antibodies. The sdAbs may be derived from any species including, but not limited to mouse, rat, human, camel, llama, lamprey, fish, shark, goat, rabbit, and bovine. Single-domain antibodies contemplated herein also include naturally occurring single-domain antibody molecules from species other than Camelidae and sharks.

In some embodiments, the sdAb is derived from a naturally occurring single-domain antigen binding molecule known as heavy chain antibody devoid of light chains (also referred herein as “heavy chain-only antibodies”, or “HCAb”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a VHH to distinguish it from the conventional VH of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present application.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909. In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacement or substitution of one or more amino acid residues in the amino acid sequence of a (naturally occurring) VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description herein. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339: 285-290, 1994; Davies and Riechmann Protein Engineering 9 (6): 531-537, 1996; Riechmann J. Mol. Biol. 259: 957-969, 1996; and Riechmann and Muyldermans J. Immunol. Meth. 231: 25-38, 1999).

In some embodiments, the sdAb is a human sdAb produced by transgenic mice or rats expressing human heavy chain segments. See, e.g., US20090307787A1, U.S. Pat. No. 8,754,287, US20150289489A1, US20100122358A1, and WO2004049794.

In some embodiments, naturally occurring VHH domains against a particular antigen or target, can be obtained from (naive or immune) libraries of Camelid VHH sequences. Such methods may or may not involve screening such a library using said antigen or target, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from (naive or immune) VHH libraries may be used, such as VHH libraries obtained from (naive or immune) VHH libraries by techniques such as random mutagenesis and/or CDR shuffling, as for example described in WO 00/43507.

In some embodiments, the sdAbs are generated from conventional four-chain antibodies. See, for example, EP 0 368 684, Ward et al. (Nature 1989 Oct. 12; 341 (6242): 544-6), Holt et al., Trends Biotechnol., 2003, 21(11):484-490; WO 06/030220; and WO 06/003388.

### Exemplary Anti-Opioid Constructs and Elements Thereof

In some embodiments, the isolated anti-opioid constructs of the invention comprise a single-domain antibody (sdAb) moiety specifically recognizing an opioid selected from the group consisting of fentanyl and carfentanil. In some embodiments, the sdAb moiety of the invention comprises a CDR1, a CDR2, and/or a CDR3. In some embodiments, the sdAb moiety comprises a CDR1, a CDR2, and a CDR3.

In some embodiments, the CDR1 comprises an amino acid sequence of GDTFGITVIG (SEQ ID NO: 1), GFTLSTLDMR (SEQ ID NO: 2), GFDFSRYDMG (SEQ ID NO: 3), GSX¹X²RVNAMG (SEQ ID NO: 4) wherein X¹ is T or D and X² is S or Y, GNTFSINSMA (SEQ ID NO: 5), or a variant of any of the foregoing comprising up to about 3 (such as about any of 1, 2, or 3) amino acid substitutions.

In some embodiments, the CDR2 comprises an amino acid sequence of ARIYNTGTVK (SEQ ID NO: 6), STVSPDGNTY (SEQ ID NO: 7), SGIKSGGGPTV (SEQ ID NO: 8), AAIDRSGATV (SEQ ID NO: 9), AELHKTDRPGSVAAN (SEQ ID NO: 10), or a variant of any of the foregoing comprising up to about 3 (such as about any of 1, 2, or 3) amino acid substitutions.

In some embodiments, the CDR3 comprises an amino acid sequence of AMGEADLTHYDLW (SEQ ID NO: 11), ADISIAAHEGGLPYW (SEQ ID NO: 12), DPDDHSGSYWGDYW (SEQ ID NO: 13), SGVLGSW (SEQ ID NO: 14), ATGLWRDHEVW (SEQ ID NO: 15), or a variant of any of the foregoing comprising up to about 3 (such as about any of 1, 2, or 3) amino acid substitutions.

In some embodiments, the CDR1 comprises an amino acid sequence of GDTFGITVIG (SEQ ID NO: 1) or a variant thereof comprising up to about 3 (such as about any of 1, 2, or 3) amino acid substitutions, the CDR2 comprises an amino acid sequence of ARIYNTGTVK (SEQ ID NO: 6) or a variant thereof comprising up to about 3 (such as about any of 1, 2, or 3) amino acid substitutions, and the CDR3 comprises an amino acid sequence of AMGEADLTHYDLW (SEQ ID NO: 11) or a variant thereof comprising up to about 3 (such as about any of 1, 2, or 3) amino acid substitutions.

In some embodiments, the CDR1 comprises an amino acid sequence of GFTLSTLDMR (SEQ ID NO: 2) or a variant thereof comprising up to about 3 (such as about any of 1, 2, or 3) amino acid substitutions, the CDR2 comprises an amino acid sequence of STVSPDGNTY (SEQ ID NO: 7) or a variant thereof comprising up to about 3 (such as about any of 1, 2, or 3) amino acid substitutions, and the CDR3 comprises an amino acid sequence of ADISIAAHEGGLPYW (SEQ ID NO: 12) or a variant thereof comprising up to about 3 (such as about any of 1, 2, or 3) amino acid substitutions.

In some embodiments, the CDR1 comprises an amino acid sequence of GFDFSRYDMG (SEQ ID NO: 3) or a variant thereof comprising up to about 3 (such as about any of 1, 2, or 3) amino acid substitutions, the CDR2 comprises an amino acid sequence of SGIKSGGGPTV (SEQ ID NO: 8) or a variant thereof comprising up to about 3 (such as about any of 1, 2, or 3) amino acid substitutions, and the CDR3 comprises an amino acid sequence of DPDDHSGSYWGDYW (SEQ ID NO: 13) or a variant thereof comprising up to about 3 (such as about any of 1, 2, or 3) amino acid substitutions.

In some embodiments, the CDR1 comprises an amino acid sequence of GSX¹X²RVNAMG (SEQ ID NO: 4) wherein X¹ is T or D and X² is S or Y or a variant thereof comprising up to about 3 (such as about any of 1, 2, or 3) amino acid substitutions, the CDR2 comprises an amino acid sequence of AAIDRSGATV (SEQ ID NO: 9) or a variant thereof comprising up to about 3 (such as about any of 1, 2, or 3) amino acid substitutions, and the CDR3 comprises an amino acid sequence of SGVLGSW (SEQ ID NO: 14) or a variant thereof comprising up to about 3 (such as about any of 1, 2, or 3) amino acid substitutions.

In some embodiments, the CDR1 comprises an amino acid sequence of GNTFSINSMA (SEQ ID NO: 5) or a variant thereof comprising up to about 3 (such as about any of 1, 2, or 3) amino acid substitutions, the CDR2 comprises an amino acid sequence of AELHKTDRPGSVAAN (SEQ ID NO: 10) or a variant thereof comprising up to about 3 (such as about any of 1, 2, or 3) amino acid substitutions, and the CDR3 comprises an amino acid sequence of ATGLWRDHEVW (SEQ ID NO: 15) or a variant thereof comprising up to about 3 (such as about any of 1, 2, or 3) amino acid substitutions.

In some embodiments, the sdAb moiety comprises an FR1, an FR2, an FR3, and an FR4.

In some embodiments, the FR1 comprises an amino acid sequence of QVQLVESGGGLVQAGESLKLSCKAS (SEQ ID NO: 16), QVQLVQSGGGLVQPGGSLRLSCAAS (SEQ ID NO: 17), DVQLVESGGGMVQPGGSLRLSCVTS (SEQ ID NO: 18), QVQLVESGGGLAQAGGSLQLSCAAS (SEQ ID NO: 19), QVQLVESGGGLVQAGESLNLSCTAS (SEQ ID NO: 20), or a variant of any of the foregoing comprising up to about 3 (such as about any of 1, 2, or 3) amino acid substitutions.

In some embodiments, the FR2 comprises an amino acid sequence of WHRQRAGQQQRELV (SEQ ID NO: 21), WFRQAPGKGFEWV (SEQ ID NO: 22), WVRQAPGKGPEWV (SEQ ID NO: 23), WYRQTPGKERELV (SEQ ID NO: 24), WYRQAPGKQRELV (SEQ ID NO: 25), or a variant of any of the foregoing comprising up to about 3 (such as about any of 1, 2, or 3) amino acid substitutions.

In some embodiments, the FR3 comprises an amino acid sequence of YADSVKGRFTLSSDSANDVVYLEMTDLKPEDTAVYYCH (SEQ ID NO: 26), YSDSAKGRFAISRDVAKNTVNLQMSSLKPEDTAVYYCN (SEQ ID NO: 27), YLDSVKGRFTVSRDNAKNTLYLQMNNLKPEDTARYYCA (SEQ ID NO: 28), YSESVRGRFTISKNDX³KX⁴IVWLQMNNLTTEDTAVYYCR (SEQ ID NO: 29) wherein X³ is A, Y, or W and X⁴ is N, D, H, or Y, YADSVKGRFTIATDNSRDTMYLQMTNLKSADTATYFCY (SEQ ID NO: 30), or a variant of any of the foregoing comprising up to about 3 (such as about any of 1, 2, or 3) amino acid substitutions.

In some embodiments, the FR4 comprises an amino acid sequence of GPGTQVTVSS (SEQ ID NO: 31), GKGTPVTVSS (SEQ ID NO: 32), GQGTLVTVSS (SEQ ID NO: 33), GLGTQVTVSS (SEQ ID NO: 34), GQGTLVTVSS (SEQ ID NO: 35), or a variant of any of the foregoing comprising up to about 3 (such as about any of 1, 2, or 3) amino acid substitutions.

In some embodiments, the FR1 comprises an amino acid sequence of QVQLVESGGGLVQAGESLKLSCKAS (SEQ ID NO: 16) or a variant thereof comprising up to about 3 (such as about any of 1, 2, or 3) amino acid substitutions, the FR2 comprises an amino acid sequence of WHRQRAGQQQRELV (SEQ ID NO: 21) or a variant thereof comprising up to about 3 (such as about any of 1, 2, or 3) amino acid substitutions, the FR3 comprises an amino acid sequence of YADSVKGRFTLSSDSANDVVYLEMTDLKPEDTAVYYCH (SEQ ID NO: 26) or a variant thereof comprising up to about 3 (such as about any of 1, 2, or 3) amino acid substitutions, and the FR4 comprises an amino acid sequence of GPGTQVTVSS (SEQ ID NO: 31) or a variant thereof comprising up to about 3 (such as about any of 1, 2, or 3) amino acid substitutions.

In some embodiments, the FR1 comprises an amino acid sequence of QVQLVQSGGGLVQPGGSLRLSCAAS (SEQ ID NO: 17) or a variant thereof comprising up to about 3 (such as about any of 1, 2, or 3) amino acid substitutions, the FR2 comprises an amino acid sequence of WFRQAPGKGFEWV (SEQ ID NO: 22) or a variant thereof comprising up to about 3 (such as about any of 1, 2, or 3) amino acid substitutions, the FR3 comprises an amino acid sequence of YSDSAKGRFAISRDVAKNTVNLQMSSLKPEDTAVYYCN (SEQ ID NO: 27) or a variant thereof comprising up to about 3 (such as about any of 1, 2, or 3) amino acid substitutions, and the FR4 comprises an amino acid sequence of GKGTPVTVSS (SEQ ID NO: 32) or a variant thereof comprising up to about 3 (such as about any of 1, 2, or 3) amino acid substitutions.

In some embodiments, the FR1 comprises an amino acid sequence of DVQLVESGGGMVQPGGSLRLSCVTS (SEQ ID NO: 18) or a variant thereof comprising up to about 3 (such as about any of 1, 2, or 3) amino acid substitutions, the FR2 comprises an amino acid sequence of WVRQAPGKGPEWV (SEQ ID NO: 23) or a variant thereof comprising up to about 3 (such as about any of 1, 2, or 3) amino acid substitutions, the FR3 comprises an amino acid sequence of YLDSVKGRFTVSRDNAKNTLYLQMNNLKPEDTARYYCA (SEQ ID NO: 28) or a variant thereof comprising up to about 3 (such as about any of 1, 2, or 3) amino acid substitutions, and the FR4 comprises an amino acid sequence of GQGTLVTVSS (SEQ ID NO: 33) or a variant thereof comprising up to about 3 (such as about any of 1, 2, or 3) amino acid substitutions.

In some embodiments, the FR1 comprises an amino acid sequence of QVQLVESGGGLAQAGGSLQLSCAAS (SEQ ID NO: 19) or a variant thereof comprising up to about 3 (such as about any of 1, 2, or 3) amino acid substitutions, the FR2 comprises an amino acid sequence of WYRQTPGKERELV (SEQ ID NO: 24) or a variant thereof comprising up to about 3 (such as about any of 1, 2, or 3) amino acid substitutions, the FR3 comprises an amino acid sequence of YSESVRGRFTISKNDX³KX⁴IVWLQMNNLTTEDTAVYYCR (SEQ ID NO: 29) wherein X³ is A, Y, or W and X⁴ is N, D, H, or Y or a variant thereof comprising up to about 3 (such as about any of 1, 2, or 3) amino acid substitutions, and the FR4 comprises an amino acid sequence of GLGTQVTVSS (SEQ ID NO: 34) or a variant thereof comprising up to about 3 (such as about any of 1, 2, or 3) amino acid substitutions.

In some embodiments, the FR1 comprises an amino acid sequence of QVQLVESGGGLVQAGESLNLSCTAS (SEQ ID NO: 20) or a variant thereof comprising up to about 3 (such as about any of 1, 2, or 3) amino acid substitutions, the FR2 comprises an amino acid sequence of WYRQAPGKQRELV (SEQ ID NO: 25) or a variant thereof comprising up to about 3 (such as about any of 1, 2, or 3) amino acid substitutions, the FR3 comprises an amino acid sequence of YADSVKGRFTIATDNSRDTMYLQMTNLKSADTATYFCY (SEQ ID NO: 30) or a variant thereof comprising up to about 3 (such as about any of 1, 2, or 3) amino acid substitutions, and the FR4 comprises an amino acid sequence of GQGTLVTVSS (SEQ ID NO: 35) or a variant thereof comprising up to about 3 (such as about any of 1, 2, or 3) amino acid substitutions.

In some embodiments, the sdAb moiety comprises an amino acid sequence having at least 90% sequence identity, at least 95% sequence identity, at least 96% sequence identity, at least 97% sequence identity, at least 98% sequence identity, at least 99% sequence identity, or 100% sequence identity to an sdAb sequence selected from the group consisting of SEQ ID NOs:36-44.

In some embodiments, the sdAb moiety comprises an amino acid sequence having at least 90% sequence identity, at least 95% sequence identity, at least 96% sequence identity, at least 97% sequence identity, at least 98% sequence identity, at least 99% sequence identity, or 100% sequence identity to an sdAb sequence selected from the group consisting of SEQ ID NOs:39 and 41-44 and comprises any one or more, any two or more, any three or more, any four or more, any five or more, any six or more, any seven or more, any eight or more, any nine or more, or each of: an alanine at a position corresponding to position 24 in the sdAb sequence; a serine or tyrosine at a position corresponding to position 29 in the sdAb sequence; a valine at a position corresponding to position 31 in the sdAb sequence; an asparagine at a position corresponding to position 32 in the sdAb sequence; a methionine at a position corresponding to position 34 in the sdAb sequence; an arginine at a position corresponding to position 53 in the sdAb sequence; a lysine at a position corresponding to position 71 in the sdAb sequence; an alanine, tyrosine, or tryptophan at a position corresponding to position 74 in the sdAb sequence; an asparagine, aspartate, or histidine at a position corresponding to position 76 in the sdAb sequence; and a valine at a position corresponding to position 78 in the sdAb sequence. In any embodiment, the amino acids on these positions can be stipulated not to be substituted.

Constructs Comprising the Anti-opioid sdAb Moiety

The anti-opioid construct comprising the anti-opioid sdAb moiety can be of any possible format.

In some embodiments, the anti-opioid construct comprising the anti-opioid sdAb moiety may further comprise additional polypeptide sequences, such as one or more antibody moieties, or Fc fragment of immunoglobulin.

In some embodiments, the additional polypeptide sequences may be a second antibody moiety (such as sdAb, scFv, full-length antibody). The second antibody moiety can recognize the same or a different antigen as the anti-opioid sdAb moiety.

In some embodiments, the additional polypeptide sequences may increase the antibody construct half-life, solubility, or absorption, reduce immunogenicity or toxicity, eliminate or attenuate undesirable side effects, and/or confer other advantageous properties to and/or reduce undesired properties of the anti-opioid construct of the invention, compared to the anti-opioid sdAb described herein per se. Some non-limiting examples of such additional polypeptide sequences are serum proteins, such as human serum albumin (see for example WO 00/27435) or haptenic molecules (for example haptens that are recognized by circulating antibodies, see for example WO 98/22141). It was shown that linking fragments of immunoglobulins (such as VH domains) to serum albumin or fragments thereof may increase antibody half-life (see e.g. WO 00/27435 and WO 01/077137). Thus, in some embodiments, the anti-opioid construct of the present invention may comprise an anti-opioid sdAb moiety described herein linked to serum albumin (or to a suitable fragment thereof), optionally via a suitable linker (such as peptide linker). In some embodiments, the anti-opioid sdAb moiety described herein can be linked to a fragment of serum albumin at least comprising serum albumin domain III. (see PCT/EP2007/002817).

### Heavy Chain-Only Antibody (HCAb)

In some embodiments, anti-opioid sdAb moiety described herein can be linked to one or more (preferably human) CH2 and/or CH3 domains, optionally via a linker sequence, to increase its half-life in vivo.

Thus in some embodiments, the anti-opioid construct is an HCAb (hereinafter referred to as “anti-opioid HCAb”) comprising an anti-opioid sdAb moiety described herein fused to an Fc fragment of an immunoglobulin, such as IgA, IgD, IgE, IgG, and IgM. In some embodiments, the anti-opioid HCAb comprises an Fc sequence of IgG, such as any of IgG1, IgG2, IgG3, or IgG4. In some embodiments, the Fc fragment is a human Fc. In some embodiments, the Fc fragment is a human IgG1 Fc. In some embodiments, the anti-opioid HCAb is monomeric. In some embodiments, the anti-opioid HCAb is dimeric. In some embodiments, the anti-opioid sdAb moiety and the Fc fragment are optionally connected by a peptide linker.

Multivalent and/or Multispecific Antibodies

In some embodiments, the anti-opioid construct comprises an anti-opioid sdAb moiety described herein fused to one or more other antibody moiety (such as an antibody moiety that specifically recognizes an opioid or another antigen). The one or more other antibody moiety can be of any antibody or antibody fragment format, such as a multispecific sdAb (such as bispecific sdAb), a full-length antibody, a Fab, a Fab′, a (Fab′)2, an Fv, a single chain Fv (scFv), an scFv-scFv, a minibody, a diabody, or a sdAb.

In some embodiments, the anti-opioid construct comprises a first anti-opioid sdAb moiety of the invention described herein fused to a second anti-opioid sdAb moiety of the invention. The first and second anti-opioid sdAb moieties can include any anti-opioid sdAb moieties described herein. The first and second anti-opioid sdAb moieties can be fused via a peptide linker.

In some embodiments, the anti-opioid construct comprising an anti-opioid sdAb moiety and one or more other antibody moiety is monospecific. In some embodiments, the anti-opioid construct comprising an anti-opioid sdAb moiety and one or more other antibody moiety is multispecific (such as bispecific). Multispecific molecules are molecules that have binding specificities for at least two different antigens or epitopes (e.g., bispecific antibodies have binding specificities for two antigens or epitopes). Multispecific molecules with more than two valencies and/or specificities are also contemplated. For example, trispecific antibodies can be prepared. Tutt et al. J. Immunol. 147: 60 (1991). It is to be appreciated that one of skill in the art could select appropriate features of individual multispecific molecules described herein to combine with one another to form a multi-specific anti-opioid molecule of the invention.

In some embodiments, the anti-opioid construct is multivalent but monospecific, i.e., the anti-opioid construct comprises an anti-opioid sdAb moiety described herein and at least a second antibody moiety specifically recognizing the same epitope as the anti-opioid sdAb moiety. In some embodiments, the one or more antibody moiety specifically recognizing the same epitope as the anti-opioid sdAb moiety described herein may comprise the same CDRs and/or the same VHH amino acid sequence as the anti-opioid sdAb moiety. For example, the anti-opioid construct may comprise two or more anti-opioid sdAb moieties described herein, wherein the two or more anti-opioid sdAb moieties are the same. In some embodiments, the anti-opioid sdAb moieties are optionally connected by peptide linker(s).

In some embodiments, the anti-opioid construct is multivalent and multispecific, i.e., the anti-opioid construct comprises an anti-opioid sdAb moiety described herein and at least a second antibody moiety specifically recognizing a second antigen other than an opioid such as fentanyl and carfentanil, or a different fentanyl or carfentanil epitope than that recognized by the anti-opioid sdAb moiety. In some embodiments, the second antibody moiety is a sdAb.

### Peptide Linkers

In some embodiments, the two or more antibody moieties within the anti-opioid construct can be optionally connected by a peptide linker. The length, the degree of flexibility and/or other properties of the peptide linker(s) used in the anti-opioid construct may have some influence on properties, including but not limited to the affinity, specificity or avidity for one or more particular antigens or epitopes. For example, longer peptide linkers may be selected to ensure that two adjacent domains do not sterically interfere with one another. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a mutated human IgG1 hinge (see SEQ ID NO: 445 of U.S. Pat. No. 11,673,954). In some embodiments, the peptide linker is a flexible linker. Exemplary flexible linkers include glycine polymers (G)ₙ, glycine-serine polymers (including, for example, (GS)ₙ, (GSGGS (SEQ ID NO: 45))ₙ, (GGGS (SEQ ID NO: 46))ₙ, and (GGGGS (SEQ ID NO: 47))ₙ, where n is an integer of at least one, such as 1, 2, 3, 4, 5, 6, 7, 8, 9, 10, 15, 20, 25 or more, and/or, optionally, up to 10, 15, 20, 25, 30, 35, or more), glycine-alanine polymers, alanine-serine polymers, and other flexible linkers known in the art. Exemplary peptide linkers can include peptide sequences such as GGGGSGGGS (SEQ ID NO: 48), GGGGSGGGGSGGGGS (SEQ ID NO: 49), GPGGP (SEQ ID NO: 50), AALVGPGGQGGGGSGGGGSGGGGSGGGGSGGGGSMA (SEQ ID NO: 51), EPKSSDKTHTSPPSP (SEQ ID NO: 52), and GPGGQGTGPGGS (SEQ ID NO: 53). Other suitable peptide linkers are provided in Klein J S, Jiang S, Galimidi R P, Keeffe J R, Bjorkman P J. Design and characterization of structured protein linkers with differing flexibilities. Protein Eng Des Sel. 2014 October;27(10):325-30.

### Anti-Opioid Antibody Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleic acid sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for substitutional mutagenesis include the HVRs and FRs. Conservative substitutions are shown in Table 1 under the heading of “Preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity.

TABLE 1
Amino acid substitutions.

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) Hydrophobic: Norleucine, Met, Ala, Val, Leu, Ile;
(2) Neutral hydrophilic: Cys, Ser, Thr, Asn, Gln;
(3) Acidic: Asp, Glu;
(4) Basic: His, Lys, Arg;
(5) Residues that influence chain orientation: Gly, Pro;
(6) Aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VHH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

In some embodiments, an anti-opioid construct provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Antibody Affinity

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

In some embodiments, the K_{d} of the binding between the anti-opioid sdAb moiety and the opioid is about 10⁻⁵ M to about 10⁻⁶ M, about 10⁻⁶M to about 10⁻⁷ M, about 10⁻⁷M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁵ M to about 10⁻¹² M, about 10⁻⁶M to about 10⁻¹² M, about 10⁻⁷M to about 10⁻¹² M, about 10⁻⁸M to about 10⁻¹² M, about 10⁻⁹ M to about 10⁻¹² M, about 10⁻¹¹ M to about 10⁻¹² M, about 10⁻⁵ M to about 10⁻¹¹ M, about 10⁻⁷ M to about 10⁻¹¹ M, about 10⁻⁸M to about 10⁻¹¹ M, about 10⁻⁹ M to about 10⁻¹¹ M, about 10⁻⁵ M to about 10⁻¹⁰ M, about 10⁻⁷M to about 10⁻¹⁰ M, about 10⁻⁸M to about 10⁻¹⁰ M, about 10⁻⁵ M to about 10⁻⁹ M, about 10⁻⁷ M to about 10⁻⁹ M, about 10⁻⁵ M to about 10⁻⁸ M, or about 10⁻⁶ M to about 10⁻⁸M.

In some embodiments, the Kₒₙ of the binding between the anti-opioid sdAb moiety and the opioid is about 10² M⁻¹s⁻¹ to about 10⁴ M⁻¹s⁻¹, about 10⁴ M⁻¹s⁻¹ to about 10⁶ M⁻¹s⁻¹, about 10⁶ M⁻¹s⁻¹ to about 107M⁻¹s⁻¹, about 10² M⁻¹s⁻¹ to about 107M⁻¹s⁻¹, about 103 M⁻¹s⁻¹ to about 10⁷M⁻¹s⁻¹, about 10⁴ M⁻¹ s⁻¹to about 10⁷M⁻¹s⁻¹, about 10⁵ M⁻¹s⁻¹ to about 10⁷ M⁻¹ s¹, about 10¹ M⁻¹ s⁻¹ to about 10⁶ M⁻¹s⁻¹, or about 10⁴ M⁻¹s⁻¹ to about 10⁶ M⁻¹s⁻¹.

In some embodiments, the K_{off}of the binding between the anti-opioid sdAb moiety and the opioid is about 1 s¹ to about 10⁻² s⁻¹, about 10⁻² s⁻¹ to about 10 s⁻¹, about 10⁻⁴s⁻¹ to about 10⁻⁵ s⁻¹, about 10⁵ s⁻¹ to about 10⁻⁶ s⁻¹, about 1 s⁻¹ to about 10⁻⁶ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵s⁻¹, or about 10⁻³ s⁻¹ to about 10⁻⁵ s⁻¹.

### Chimeric or Humanized Antibodies

In some embodiments, the anti-opioid antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a camelid species, such as llama) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences or are modified to have residues from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); Osbourn et al., Methods 36:61-68 (2005) and Klimka et al., Br. J. Cancer, 83:252-260 (2000) (describing the “guided selection” approach to FR shuffling); Vincke et al. J Biol Chem. 284(5):3273-3284 (2009); and Sulea, T. Humanization of Camelid Single-Domain Antibodies. Methods Mol. Biol. 2022, 2446, 299-312.

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, the sdAbs are modified, such as humanized, without diminishing the native affinity of the domain for antigen and while reducing its immunogenicity with respect to a heterologous species. For example, the amino acid residues of the antibody variable domain (VHH) of a llama antibody can be determined, and one or more of the Camelid amino acids, for example, in the framework regions, are replaced by their human counterpart as found in the human consensus sequence, without that polypeptide losing its typical character, i.e. the humanization does not significantly affect the antigen binding capacity of the resulting polypeptide. Humanization of Camelid single-domain antibodies can be obtained by the introduction and mutagenesis of a limited amount of amino acids in a single polypeptide chain. This is in contrast to humanization of scFv, Fab′, (Fab′)2 and IgG, which requires the introduction of amino acid changes in two chains, the light and the heavy chain and the preservation of the assembly of both chains.

Single-domain antibodies comprising a VHH domain can be humanized to have human-like sequences. In some embodiments, the FR regions of the VHH domain used herein comprise at least about any one of 50%, 60%, 70%, 80%, 90%, 95% or more of amino acid sequence homology to human VH framework regions. One exemplary class of humanized VHH domains is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanization.

Another exemplary class of humanized Camelid single-domain antibodies has been described in WO 03/035694 and contains hydrophobic FR2 residues typically found in conventional antibodies of human origin or from other species, but compensating this loss in hydrophilicity by the charged arginine residue on position 103 that substitutes the conserved tryptophan residue present in V_{H} from double-chain antibodies. As such, peptides belonging to these two classes show a high amino acid sequence homology to human V_{H} framework regions, and said peptides might be administered to a human directly without expectation of an unwanted immune response therefrom and without the burden of further humanization.

### Pharmaceutical Compositions

Further provided by the present application are pharmaceutical compositions comprising any one of the anti-opioid constructs comprising a sdAb specifically recognizing an opioid as described herein or a nucleic acid configured to express the anti-opioid construct. Vectors and nucleic acids configured to express proteins in vivo are well known in the art. Vectors include viruses such as adenoviruses, among other vectors.

The pharmaceutical compositions can optionally include a pharmaceutically acceptable carrier. Pharmaceutical compositions can be prepared by mixing an anti-opioid construct or nucleic acid described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they should be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally.

### Methods

The invention is also directed to methods of using the anti-opioid constructs or nucleic acids configured to express same. The methods can comprise administering the anti-opioid constructs or nucleic acids configured to express same to an individual to thereby bind the anti-opioid construct to an opioid present within a body of the individual. The opioid can be fentanyl and/or carfentanil, depending on the binding characteristics of the particular anti-opioid construct. The anti-opioid construct or nucleic acid configured to express same can be administered in the form of a pharmaceutical composition as described herein. The individual can be an individual who has taken or has been administered an opioid such as fentanyl and/or carfentanil. The individual can be an individual who is under the influence of an opioid such as fentanyl and/or carfentanil. The individual can be an individual who is suffering from an overdose of an opioid such as fentanyl and/or carfentanil. The anti-opioid construct or nucleic acid configured to express same can be administered in an amount effective to reduce the effects of the opioid in the individual. The anti-opioid construct or nucleic acid configured to express same can be administered in an amount effective to treat the overdose.

Dosages and desired concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy.

The anti-opioid constructs or nucleic acids can be administered in accord with known methods, such as intravenous administration as a bolus or by continuous infusion over a period of time, by intramuscular, intraperitoneal, intracerobrospinal, subcutaneous, intravenous (i.v.), intra-articular, intrasynovial, intrathecal, oral, topical, or inhalation routes. A reconstituted formulation can be prepared by dissolving a lyophilized anti-opioid construct described herein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution, Ringer's solution or dextrose solution, or aqueous solutions of salts and/or buffers.

In some embodiments, the pharmaceutical compositions can be administered to the individual by subcutaneous (i.e. beneath the skin) administration. For such purposes, the pharmaceutical compositions may be injected using a syringe. However, other devices for administration of the pharmaceutical compositions are available such as injection devices; injector pens; auto-injector devices, needleless devices; and subcutaneous patch delivery systems.

In some embodiments, the pharmaceutical compositions can be administered to the individual intravenously. In some embodiments, the pharmaceutical composition is administered to an individual by infusion, such as intravenous infusion. Infusion techniques for immunotherapy are known in the art (see, e.g., Rosenberg et al., New Eng. J. of Med. 319: 1676 (1988)).

The individual can be an animal, such as a mammal or a human.

### Definitions

As used herein, “treatment” or “treating” is an approach for obtaining beneficial or desired results, including clinical results. For purposes of this invention, beneficial or desired clinical results include but are not limited to one or more of the following: alleviating one or more symptoms resulting from a condition, diminishing the extent of a condition, stabilizing a condition (e.g., avoiding or delaying the worsening of the disease), delaying or slowing the progression of a condition, ameliorating a condition state, decreasing the dose of one or more other medications required to treat the condition, increasing the quality of life, and/or prolonging survival. Also encompassed by “treatment” is a reduction of a pathological consequence of a condition. The methods of the invention contemplate any one or more of these aspects of treatment. An exemplary condition is being under the influence of an opioid, such as fentanyl and carfentanil, such as suffering from an overdose of the opioid.

The term “therapeutically effective amount” used herein refers to an amount of an agent, a combination of agents, or a pharmaceutical composition comprising such agents sufficient to treat a specified disorder, condition, or disease, such as to ameliorate, palliate, lessen, and/or delay one or more of its symptoms.

The term “antibody” or “antibody moiety” is used in the broadest sense and encompasses various antibody structures, including but not limited to monoclonal antibodies, polyclonal antibodies, multi specific antibodies (e.g., bispecific antibodies), full-length antibodies and antigen-binding fragments thereof, so long as they exhibit the desired antigen-binding activity.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs” (variable domain of the heavy chain of the heavy chain antibody). Some VHHs can also be known as nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

An “isolated” antibody (or construct) is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody (or construct) includes the antibody in situ within recombinant cells since at least one component of the antibody's natural environment will not be present. Ordinarily, however, an isolated polypeptide, antibody, or construct will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH”. VHH is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

“Humanized” forms of non-human (e.g., llama or camelid) antibodies are antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs, and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” is used to refer to hypervariable regions as defined by the Kabat system. See Kabat et al., Sequences of Proteins of Immunological Interest, 5th Ed. Public Health Service, National Institutes of Health, Bethesda, Md. (1991).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table 2.

TABLE 2
HVR delineations.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The amino acid residues of a single-domain antibody (such as VHH) can be numbered according to the general numbering for VH domains given by Kabat et al. (“Sequence of proteins of immunological interest”, US Public Health Services, NIH Bethesda, Md., Publication No. 91), as applied to VHH domains from Camelids in the article of Riechmann and Muyldermans, J. Immunol. Methods 2000 Jun. 23; 240 (1-2): 185-195. According to this numbering, FR1 of a VHH comprises the amino acid residues at positions 1-30, CDR1 of a VHH comprises the amino acid residues at positions 31-35, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. In this respect, it should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering).

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the VH, the subgroup may be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

As used herein, the term “specifically binds,” “specifically recognizes,” or is “specific for” refers to measurable and reproducible interactions such as binding between a target and an antigen binding protein (such as a sdAb), which is determinative of the presence of the target in the presence of a heterogeneous population of molecules, including biological molecules. For example, an antigen binding protein (such as a sdAb) that specifically binds a target (which can be an epitope) is an antigen binding protein (such as a sdAb) that binds this target with greater affinity, avidity, more readily, and/or with greater duration than it binds other targets. In some embodiments, the extent of binding of an antigen binding protein (such as a sdAb) to an unrelated target is less than about 10% of the binding of the antigen binding protein (such as sdAb) to the target as measured, e.g., by a radioimmunoassay (RIA). In some embodiments, an antigen binding protein (such as a sdAb) that specifically binds a target has a dissociation constant (K_{d}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (such as a sdAb) for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. The term “monospecific” as used herein denotes an antigen binding protein (such as a sdAb) that has one or more binding sites each of which bind the same epitope of the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The dissociation constant (K_{D} or K_{d}) is used as an indicator showing affinity of antibodies to antigens. For example, easy analysis is possible by the Scatchard method using antibodies marked with a variety of marker agents, as well as by using BiacoreX (made by Amersham Biosciences), which is an over-the-counter, measuring kit, or similar kit, according to the user's manual and experiment operation method attached with the kit. The K_{D} value that can be derived using these methods is expressed in units of M (Mols). An antibody or antigen-binding fragment thereof that specifically binds to a target may have a dissociation constant (K_{d}) of, for example, ≤10⁻⁵ M, ≤10⁻⁶ M, ≤10⁻⁷ M, ≤10⁻⁸ M, ≤10⁻⁹ M, ≤10⁻¹⁰ M, ≤10⁻¹⁰ M, 10⁻¹¹ M, or 10⁻¹² M.

Binding specificity of the antibody or antigen-binding domain can be determined experimentally by methods known in the art. Such methods comprise, but are not limited to Western blots, ELISA-, RIA-, ECL-, IRMA-, EIA-, BIAcore-tests and peptide scans.

Half maximal inhibitory concentration (IC₅₀) is a measure of the effectiveness of a substance (such as an antibody) in inhibiting a specific biological or biochemical function. It indicates how much of a particular drug or other substance (inhibitor, such as an antibody) is needed to inhibit a given biological process by half. The values are typically expressed as molar concentration. IC₅₀ is comparable to an EC₅₀ for agonist drug or other substance (such as an antibody). EC₅₀ also represents the plasma concentration required for obtaining 50% of a maximum effect in vivo. As used herein, an “IC₅₀” is used to indicate the effective concentration of an antibody (such as an anti-opioid sdAb) needed to neutralize 50% of the antigen bioactivity (such as opioid bioactivity) in vitro. IC₅₀ or EC₅₀ can be measured by bioassays such as inhibition of ligand binding by FACS analysis (competition binding assay), cell based cytokine release assay, or amplified luminescent proximity homogeneous assay (AlphaLISA).

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting” and/or “consisting essentially of” embodiments.

The term “about X-Y” used herein has the same meaning as “about X to about Y.”

Additional definitions of can be found in U.S. Pat. No. 11,673,954, which is incorporated herein by reference. In case of conflicts, the definitions provided explicitly herein control.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Introduction

Here, we describe the identification of novel VHHs for fentanyl using a naive VHH phage display library screened against a fentanyl hapten. Our lead VHH (JGFN4) was specific for fentanyl and displayed no cross-reactivity with the closely related synthetic opioid carfentanil. The crystal structure of JGFN4 in the presence of fentanyl revealed a homodimer with each protomer bound to one fentanyl molecule. Interestingly, the homodimer formed as a result of reciprocal domain swapping between CDR3 at the C-terminal end of each protomer. In atypical fashion, CDR3 did not form key interactions with fentanyl, rather binding was through CDR1 and CDR2. Through rational mutagenesis and additional crystallographic analysis, we identified a mutation that allowed monomeric JGFN4 to bind fentanyl abrogating the need for domain swapping. Biolayer interferometry (BLI) and differential scanning fluorimetry (DSF) determined that mutant versions of JGFN4 had increased affinity for fentanyl compared to wild type JGFN4. Taken together, our study documents an important proof-of-concept showing that single-domain antibodies can engage synthetic opioids, thus warranting further investigation of these domains as a class of therapeutics for treating OUD.

### Methods

### Haptens

Fentanyl-based haptens F₁ and F₃ and their conjugates used for isolation or characterization of anti-fentanyl VHH were obtained from Dr. Pravetoni. The structure and synthesis of F₁-biotin for BLI, F₃-biotin for biopanning, and F₁/F₃-BSA for ELISA was previously described.³³⁻³⁵

### VHH Identification

Using a previously described high diversity (>10¹⁰) camelid VHH phage display library a biopanning campaign against fentanyl and carfentanil haptens was carried out^{30,36}. Briefly, the library was incubated overnight with 1 μg of a fentanyl haptens conjugated with biotin. The bound phage and biotinylated fentanyl hapten were then incubated at room temperature with magnetic Dynabeads, M-270 Streptavidin (Thermo Fisher Scientific) for 1 h. The resulting phage-opioid-bead complexes were washed with PBST and PBS before the remaining phage were eluted with 1 mL of 100 mM triethylamine for 8 minutes before being neutralized with 500 ul of 1M Tris-HCl, pH 7.5. Eluted phage were then used to infect TG1 E. coli for 1 h at 37° C. before being plated on 2xYT agar plates (containing Ampicillin and Glucose, AG) and grown overnight at 37° C. The following day the infected TG1 growths were collected and resuspended in 36 mL of 2xYT-AG media. To generate more phage the TG1 growth was used to inoculate a new 100-mL culture and grown at 37° C. with 250 rpm until reaching an OD₆₀₀=0.8. Once reaching the desired OD₆₀₀, the phage-infected TG1 bacteria was co-infected with M13K07 helper phage and grown overnight at 30° C. with 250 rpm. The next day, the media from the coinfected culture was harvested, and phage were purified with standard PEG precipitation methods³⁷. Following round 3, the eluted phage was also used to infect SS320 E. coli and plated in a dilution series to obtain single colonies growths. The single colonies were then collected, grown, and induced with 1 mM isopropyl-beta-D-thiogalactopyranoside. The supernatant media containing the induced camelid antibodies was evaluated using a previously described ELISA method to identify strong binders against the fentanyl hapten and an additional ELISA was performed to evaluate any cross-reactivity with a carfentanil hapten³⁶⁻³⁸. Strong binding colonies were isolated and purified for further studies described below and sequenced to identify unique antibody leads.

### VHH Monomer Constructs

VHH constructs included the PelB leader sequence (MKYLLPTAAAGLLLLAAQPAMA (SEQ ID NO: 54)) followed by a VHH sdAb sequence (JGFN1, JGFN2, JGFN3, JGFN4, JGFN5, or a JGFN4 mutant, see Tables 4-7), followed by a 6x His tag (GPGGQHHHHHH (SEQ ID NO: 55)), followed by an HA tag (GAYPYDVPDYAS (SEQ ID NO: 56)). Sequences of the VHH constructs containing JGFN1, JGFN2, JGFN3, JGFN4, and JGFN5 are below:

JGFN1 VHH Construct (nucleic acid)
(SEQ ID NO: 57)
ATGAAATACCTATTGCCTACGGCGGCCGCTGGATTGTTATTACTCGCG
GCCCAGCCGGCCATGGCTCAGGTGCAGCTCGTGGAGTCTGGGGGAGGC
TTGGTGCAGGCTGGAGAGTCTCTGAAACTCTCCTGTAAGGCCTCTGGA
GATACCTTCGGTATCACCGTCATCGGCTGGCACCGCCAACGTGCAGGT
CAACAGCAGCGCGAATTGGTCGCGCGCATTTATAATACTGGGACTGTA
AAATACGCCGACTCCGTGAAGGGCCGGTTCACCCTCTCCAGTGACAGC
GCCAACGACGTGGTGTATCTGGAGATGACTGACTTGAAACCGGAAGAC
ACGGCCGTCTATTACTGCCACGCGATGGGCGAAGCTGACCTCACTCAC
TATGACCTCTGGGGCCCGGGGACCCAGGTCACCGTCTCCAGCGGCCCG
GGAGGCCAACACCATCACCACCATCATGGCGCATATCCGTATGATGTG
CCGGACTATGCTTCTTAG
JGFN1 VHH Construct (protein)
(SEQ ID NO: 58)
MKYLLPTAAAGLLLLAAQPAMAQVQLVESGGGLVQAGESLKLSCKASG
DTFGITVIGWHRQRAGQQQRELVARIYNTGTVKYADSVKGRFTLSSDS
ANDVVYLEMTDLKPEDTAVYYCHAMGEADLTHYDLWGPGTQVTVSSGP
GGQHHHHHHGAYPYDVPDYAS
JGFN2 VHH Construct (nucleic acid)
(SEQ ID NO: 59)
ATGAAATACCTATTGCCTACGGCGGCCGCTGGATTGTTATTACTCGCG
GCCCAGCCGGCCATGGCTCAGGTGCAGCTCGTGCAGTCTGGGGGAGGC
TTGGTGCAACCTGGGGGTTCTCTGAGACTCTCCTGTGCAGCCTCTGGC
TTCACCCTCAGTACGTTGGATATGAGGTGGTTCCGCCAGGCTCCAGGA
AAGGGGTTCGAGTGGGTCTCAACTGTTAGTCCTGATGGTAACACGTAC
TACTCAGACTCCGCGAAGGGCCGATTCGCCATCTCCAGAGACGTCGCC
AAGAACACGGTGAATCTGCAAATGAGCAGCCTGAAACCTGAAGACACG
GCCGTCTATTATTGTAATGCAGATATTTCAATCGCGGCGCACGAGGGC
GGACTGCCCTACTGGGGCAAAGGGACCCCGGTCACCGTCTCCAGCGGC
CCGGGAGGCCAACACCATCACCACCATCATGGCGCATATCCGTATGAT
GTGCCGGACTATGCTTCTTAG
JGFN2 VHH Construct (protein)
(SEQ ID NO: 60)
MKYLLPTAAAGLLLLAAQPAMAQVQLVQSGGGLVQPGGSLRLSCAASG
FTLSTLDMRWFRQAPGKGFEWVSTVSPDGNTYYSDSAKGRFAISRDVA
KNTVNLQMSSLKPEDTAVYYCNADISIAAHEGGLPYWGKGTPVTVSSG
PGGQHHHHHHGAYPYDVPDYAS
JGFN3 VHH Construct (nucleic acid)
(SEQ ID NO: 61)
ATGAAATACCTATTGCCTACGGCGGCCGCTGGATTGTTATTACTCGCG
GCCCAGCCGGCCATGGCTGATGTTCAGCTCGTGGAGTCTGGGGGAGGC
ATGGTGCAGCCTGGGGGGTCTCTGAGACTCTCCTGTGTAACCTCTGGA
TTCGACTTCAGCCGCTATGATATGGGCTGGGTCCGCCAGGCTCCAGGG
AAGGGGCCCGAGTGGGTCTCAGGAATTAAAAGTGGTGGCGGTCCCACA
GTCTACCTAGACTCCGTGAAGGGCCGATTCACCGTCTCCAGAGATAAC
GCCAAGAATACTTTATATCTCCAAATGAACAACCTGAAACCTGAGGAC
ACGGCCCGGTATTACTGTGCGAGAGATCCCGACGACCACAGTGGTAGT
TACTGGGGCGACTACTGGGGCCAGGGGACCCTGGTCACCGTCTCCAGC
GGCCCGGGAGGCCAACACCATCACCACCATCATGGCGCATATCCGTAT
GATGTGCCGGACTATGCTTCTTAG
JGFN3 VHH Construct (protein)
(SEQ ID NO: 62)
MKYLLPTAAAGLLLLAAQPAMADVQLVESGGGMVQPGGSLRLSCVTSG
FDFSRYDMGWVRQAPGKGPEWVSGIKSGGGPTVYLDSVKGRFTVSRDN
AKNTLYLQMNNLKPEDTARYYCARDPDDHSGSYWGDYWGQGTLVTVSS
GPGGQHHHHHHGAYPYDVPDYAS
JGFN4 VHH Construct (nucleic acid)
(SEQ ID NO: 63)
ATGAAATACCTATTGCCTACGGCGGCCGCTGGATTGTTATTACTCGCG
GCCCAGCCGGCCATGGCTCAGGTGCAGCTCGTGGAGTCCGGGGGAGGC
TTGGCGCAGGCTGGGGGATCTCTGCAACTCTCGTGCGCAGCTTCCGGT
AGTACCTCCAGAGTCAATGCCATGGGTTGGTACCGCCAGACTCCGGGG
AAAGAGCGCGAGTTGGTCGCGGCGATTGACCGCTCCGGAGCCACGGTC
TACTCGGAGTCCGTGAGGGGACGATTCACCATCTCCAAAAACGATGCC
AAAAACATCGTGTGGCTGCAAATGAACAACCTGACGACTGAAGATACG
GCCGTCTATTACTGTCGCAGTGGGGTCCTTGGTTCCTGGGGCCTGGGG
ACCCAGGTCACCGTCTCCAGCGGCCCGGGAGGCCAACACCATCACCAC
CATCATGGCGCATATCCGTATGATGTGCCGGACTATGCTTCTTAG
JGFN4 VHH Construct (protein)
(SEQ ID NO: 64)
MKYLLPTAAAGLLLLAAQPAMAQVQLVESGGGLAQAGGSLQLSCAASG
STSRVNAMGWYRQTPGKERELVAAIDRSGATVYSESVRGRFTISKNDA
KNIVWLQMNNLTTEDTAVYYCRSGVLGSWGLGTQVTVSSGPGGQHHHH
HHGAYPYDVPDYAS
JGFN5 VHH Construct (nucleic acid)
(SEQ ID NO: 65)
ATGAAATACCTATTGCCTACGGCGGCCGCTGGATTGTTATTACTCGCG
GCCCAGCCGGCCATGGCTCAGGTGCAGCTCGTGGAGTCTGGGGGAGGC
TTGGTGCAGGCTGGAGAATCTCTGAATCTCTCCTGCACAGCCTCCGGG
AACACCTTCAGTATCAATTCCATGGCCTGGTACCGCCAGGCTCCAGGA
AAGCAGCGCGAGTTGGTCGCGGAACTTCATAAAACTGATCGGCCAGGT
AGTGTCGCCGCGAACTATGCAGACTCCGTGAAGGGCCGATTCACCATC
GCCACCGACAACTCCAGAGACACGATGTATCTCCAAATGACCAACCTA
AAATCTGCGGATACTGCCACCTATTTCTGTTATGCGACGGGGCTCTGG
AGGGATCATGAAGTTTGGGGCCAGGGCACCCTGGTCACCGTCTCCAGC
GGCCCGGGAGGCCAACACCATCACCACCATCATGGCGCATATCCGTAT
GATGTGCCGGACTATGCTTCTTAG
JGFN5 VHH Construct (protein)
(SEQ ID NO: 66)
MKYLLPTAAAGLLLLAAQPAMAQVQLVESGGGLVQAGESLNLSCTASG
NTFSINSMAWYRQAPGKQRELVAELHKTDRPGSVAANYADSVKGRFTI
ATDNSRDTMYLQMTNLKSADTATYFCYATGLWRDHEVWGQGTLVTVSS
GPGGQHHHHHHGAYPYDVPDYAS

### Generation of Mammalian Expression Vectors

Fentanyl-binding VHH homodimer expression vectors include an open reading frame (ORF) that initiates with a murine IGHV signal peptide (MGWSCIILFLVATATGVHS), followed by two copies of the VHH encoding gene linked together with a (Gly₄Ser)₄ polypeptide linker and a H-terminal 6xHis tag for affinity purification. To facilitate expression vector assembly, codon optimized gene fragments encoding VHH homodimer were synthesized (Twist Bioscience) and cloned into pcDNA3.4 expression plasmids via Gibson assembly²⁶.

### Mammalian Expression and Purification

Fentanyl-binding VHH homodimers were produced via transient expression with the Expi293 expression system according to manufacturer instructions (ThermoFisher Catalog #A14635). Cell culture supernatant was harvested 7 days following transfection, and VHH homodimers were purified via liquid chromatography on an AKTA pure (Cytiva) with a HisTrap excel column (Cytiva Product #29048586). The chromatographic process included column equilibration in 20 mM NaPO₄, 0.5 M NaCl, pH 7.4 followed by sample loading and a wash step with 20 mM NaPO₄, 0.5 M NaCl, 10 mM imidazole, pH 7.4 to remove non-specifically bound protein contaminants. His-tagged VHH homodimer eluted during the isocratic application of 20 mM NaPO₄, 0.5 M NaCl, 500 mM imidazole, pH 7.4. Eluate fractions were pooled and buffer exchanged into PBS, pH 7.4. Protein concentration was determined by absorbance at 280 nm on a Nanodrop and purity analysis was performed by SDS-PAGE.

### X-Ray Crystallography Studies

JGFN4 used in the structural studies was expressed with a C-terminal 6xHis-tag in SHuffle T7 Express E. coli strain (NEB) and purified over Ni-NTA Superflow (Qiagen) and Superdex 75 (Cytiva) chromatography columns. Discrete peaks corresponding to nanobody monomers and dimers were obtained. Each species was concentrated by ultrafiltration in 20 mM Tris-HCl, pH 7.4, 0.5 M NaCl, flash-frozen in liquid nitrogen, and stored at −80° C. Protein concentrations were determined based on UV absorbance at 280 nm and the theoretical extinction coefficient. JGFN4 monomer or dimer at −12 mg ml-¹ was mixed with a 5-fold molar excess of fentanyl and subjected to crystallization screening in the sitting drop vapor diffusion mode at ambient temperature, by mixing 0.1 μl each of the complex and reservoir solutions. Crystals used in the structural studies were obtained with the reservoir solutions of 0.1 M Tris-HCl, pH 8.0, 0.2 M LiCl, 20% (w/v) PEG 6000 for the crystal in space group C222₁ (from dimeric JGFN4), and 0.2 M sodium acetate, 0.1 M sodium cacodylate pH 6.5, 30% (w/v) PEG 8000, for the crystal in space group P2₁2₁2 (from monomeric JGFN4), respectively. JGFN4 monomer without fentanyl was crystallized with the reservoir solution: 20% (w/v) PEG MME 2000, 0.1 M Tris-HCl pH 8.5, 0.2 M trimethylamine N-oxide. JGFN4 N76D was purified and subjected to crystallization screening as above. Crystals of monomeric JGFN4 N76D bound to fentanyl were obtained with the reservoir solution containing 0.1 M Tris-Bicine, pH 8.5, 0.02 M each of monosaccharides (D-glucose; D-mannose; D-galactose; L-fucose; D-xylose; N-acetyl-D-glucosamine), 20% (v/v) PEG 500 MME, 10% (w/v) PEG 20000. Crystals of dimeric JGFN4 N76D bound to fentanyl were obtained with the reservoir solution containing 0.2 M sodium acetate, 20% (w/v) PEG3350. The crystals were cryo-protected by a brief soaking in the respective reservoir solution supplemented with 25% ethylene glycol and flash-cooled by plunging in liquid nitrogen. X-ray diffraction data were collected at the Advanced Photon Source NE-CAT beamline 24-ID-C and 24-ID-E. All X-ray diffraction data were processed using XDS³⁹. Initial phases were obtained by molecular replacement with PHASER⁴⁰ using Gβγ nanobody (PDB ID 6B20)⁴¹ as the search model. Model building and refinement were done using COOT and PHENIX, respectively^{42,43}. The summary of data collection and model refinement statistics is shown in Table 3.

TABLE 3
Crystallization data collection and refinement statistics.

| | | | | | Preformed | | N76D | | N76D | |
|---|---|---|---|---|---|---|---|---|---|---|
| | | | | | dimer- | | monomer- | | dimer- | |
| | Dimer-Fentanyl | | Monomer-APO | | Fentanyl | | Fentanyl | | Fentanyl | |
| Data collection | | | | | | | | | | |
| Space group | P2₁2₁2 | | C2 | | C222₁ | | C2 | | C222₁ | |
| Unit cell | | | | | | | | | | |
| dimensions | | | | | | | | | | |
| a, b, c(Å) | 85.43, 97.14, 57.58 | | 91.24, 26.86, 36.96 | | 44.79, 109.07, 50.20 | | 91.10, 26.36, 37.46 | | 57.31, 83.85, 96.61 | |
| α, β, γ(°) | 90, 90, 90 | | 90, 106.99, 90 | | 90, 90, 90 | | 90, 107.19, 90 | | 90, 90, 90 | |
| Resolution (Å) | 47.8-1.68 | (1.74-1.68) | 43.6-1.76 | (1.81-1.76) | 36.9-1.55 | (1.58-1.55) | 43.51-1.60 | (1.63-1.60) | 47.3-1.85 | (1.89-1.85) |
| R_{sym} or R_{merge} | 0.0717 | (1.04) | 0.061 | (0.279) | 0.099 | (1.63) | 0.094 | (1.94) | 0.050 | (0.839) |
| I/σI | 9.80 | (1.26) | 10.4 | (2.5) | 8.14 | (0.78) | 4.80 | (0.60) | 19.41 | (2.30) |
| Completeness (%) | 97.43 | (97.74) | 91.40 | (59.7) | 97.41 | (91.89) | 97.7 | (97.9) | 99.20 | (99.25) |
| Redundancy | 3.7 | (3.7) | 3.6 | (2.2) | 4.3 | (3.3) | 2.7 | (2.7) | 7.6 | (7.6) |
| CC_{1/2} | 0.996 | (0.677) | 0.998 | (0.901) | 0.996 | (0.436) | 0.965 | (0.450) | 1.000 | (0.875) |
| Refinement | | | | | | | | | | |
| Resolution (A) | 47.8-1.68 | (1.74-1.68) | 35.4-1.76 | (1.82-1.76) | 36.9-1.55 | (1.61-1.55) | 35.8-1.6 | (1.66-1.60) | 47.3-1.85 | (1.92-1.85) |
| No. Reflections | 53990 | (5330) | 7971 | (532) | 17815 | (1643) | 11139 | (1059) | 20120 | (1976) |

| | | | | | |
|---|---|---|---|---|---|
| R_{work}/R_{free} | 0.200/0.239 | 0.191/0.232 | 0.187/0.218 | 0.234/0.267 | 0.203/0.240 |
| No. atoms | 3921 | 918 | 1016 | 908 | 1867 |
| Protein | 3406 | 857 | 852 | 845 | 1695 |
| Ligand/ion | 104 | 0 | 29 | 25 | 68 |
| Water | 411 | 61 | 135 | 38 | 104 |
| B-factor | 34.97 | 20.92 | 28.70 | 39.07 | 44.83 |
| Protein | 33.93 | 20.73 | 27.66 | 38.99 | 44.83 |
| Ligand/ion | 40.09 | n.a. | 23.78 | 42.26 | 44.73 |
| Water | 42.34 | 23.56 | 36.36 | 38.86 | 45.48 |
| R.m.s. deviations | | | | | |
| Bond lengths (Å) | 0.015 | 0.003 | 0.003 | 0.002 | 0.008 |
| Bond angles (°) | 1.30 | 0.60 | 0.56 | 0.495 | 0.94 |

Statistics for the highest-resolution shell are shown in parentheses.

### Biolayer Interferometry

Affinity of VHH homodimer was evaluated by biolayer interferometry (BLI) using an Octet Red 96e (Sartorius) with streptavidin-coated biosensors (Sartorius Catalog #18-5020). F₁-biotin was loaded onto biosensors at 0.2 μg/mL in phosphate buffered saline with 0.1% bovine serum albumin (BSA) for 60 sec. Association of VHH homodimer to immobilized hapten was measured over the course of 5 min at VHH homodimer concentrations of 100, 250, and 500 nM, followed by dissociation in phosphate buffered saline with 0.1% BSA for 5 min. All kinetic parameters including on-rate (kₒₙ), off-rate (k_{off}), and K_{D} (k_{off}/kₒₙ) were calculated using Octet software (Sartorius).

### Competitive ELISA

Relative affinity of VHH for fentanyl was determined by competitive ELISA essentially as described (Baehr et al, doi: 10.1124/jpet.120.000124). Briefly, high-binding polystyrene 96-well assay plates (Corning) were coated with 0.05 μg/mL F₁-BSA in carbonate-bicarbonate buffer, pH 9.5 (Thermo) overnight, and blocked with 1% porcine gelatin (Sigma) in PBS-T buffer (ThermoFisher 28352) for 1 hour. Plates were loaded with free fentanyl in PBS-T, and VHH homodimer samples were applied and incubated for 2 hours. Plates were incubated with HRP-labeled anti-histidine tag secondary antibody (Santa Cruz Biotechnology Cat #sc-53073) overnight. HRP activity was quantitated with SigmaFAST OPD substrate (Sigma), and relative affinity of VHH for fentanyl was measured as concentration of fentanyl reducing OPD signal by 50% (IC₅₀).

### Differential Scanning Fluorimetry

Affinity of VHH was assessed using differential scanning fluorimetry (DSF) to measure ligand-induced stabilization of protein unfolding with Protein Thermal Shift™ Dye Kit (ThermoFisher Catalog #4461146). VHH homodimer (3 μM) was incubated in the presence of 0-1 mM fentanyl and subjected to a 25-95° C. thermal ramp at a rate of 0.015° C./see using a QuantStudio 3 RT-PCR system (ThermoFisher). Fluoresence data was captured during the thermal ramp using the system's X⁴/M4 optical filter settings. Melting temperature data was analyzed with Protein Thermal Shift™ Software v1.4 (ThermoFisher Catalog #4466038), and Boltzmann Tₘ data for each sample was obtained. Affinity of VHH homodimers and scFv (+) control for fentanyl was calculated from the concentration-dependent increase in Boltzmann Tₘ according to the method described in Kirley et al.⁴⁴.

### Results

Identification of a VHH specific for fentanyl

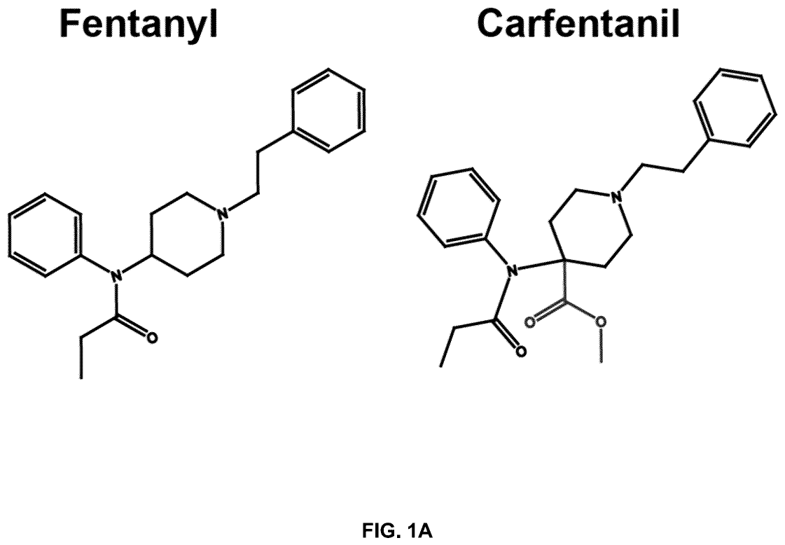

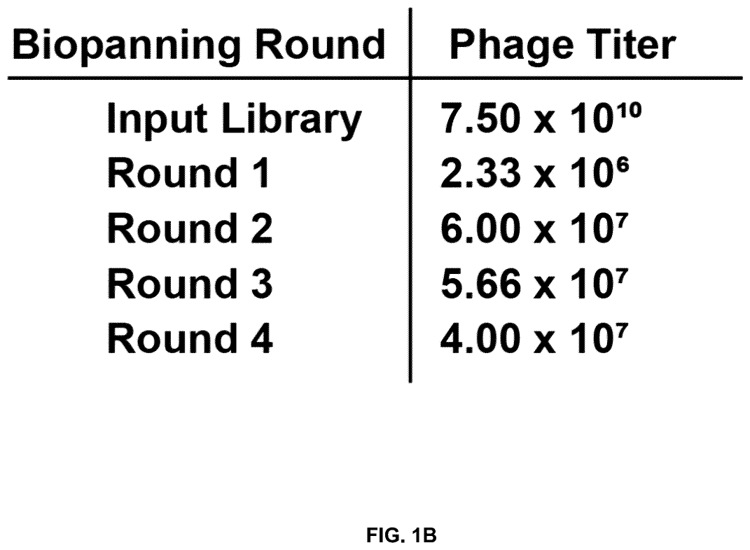

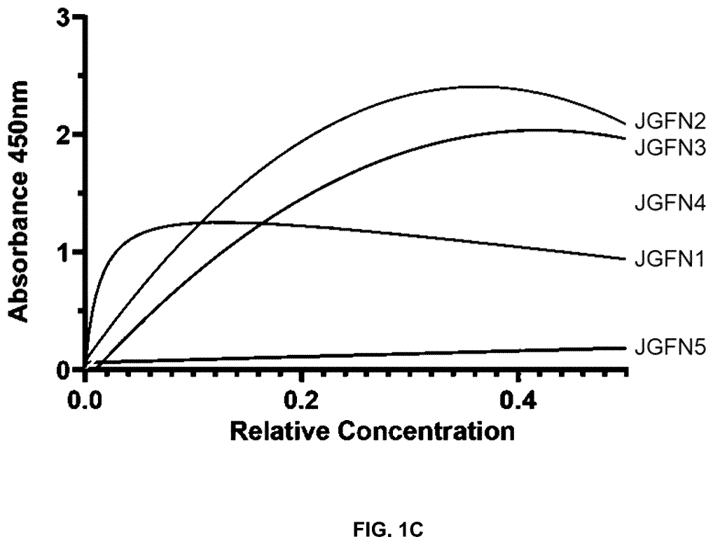

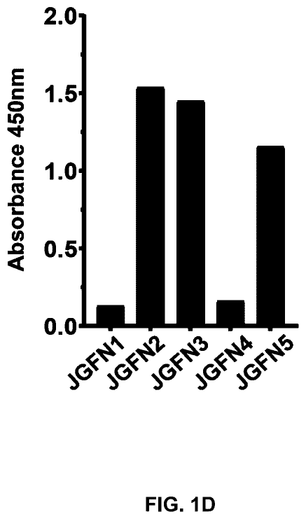

To identify a fentanyl-specific VHH, we screened our in-house camelid VHH phage display library against immobilized fentanyl hapten F3 on magnetic beads. The initial round of biopanning resulted in a large reduction in phage particles as compared to the starting library, indicative of a successful reduction of non-binding VHH variants. The second round of biopanning yielded an increased phage titer suggesting there was an enrichment of opioid binding antibodies from the round one phage. The last two rounds of biopanning conserved this phage titer and no indication of a diversity collapse was seen in the final rounds of biopanning (FIG. 1B). The supernatant of individual VHH clones induced in monoculture following rounds 3 and 4 totaling 672 were tested by ELISA for binding to fentanyl haptens. Clones that demonstrated a high ELISA signal (>0.75) were sent for sequencing. Sequencing identified a total of 5 unique camelid VHH sequences, denoted as JGFN 1-5 (Tables 4-7), that had varying affinities and selectivity for fentanyl and carfentanil. Carfentanil is a highly potent synthetic opioid that differs from fentanyl by the addition of a methyl ester to the piperidine ring. Evaluation of these five lead VHHs for cross-reactivity against the screened fentanyl hapten F3 and a homologous carfentanil hapten (F11) found that JGFN 1 and JGFN 4 were fentanyl specific while JGFN 2 and JGFN 3 showed specific binding to both fentanyl and carfentanil. Interestingly JGFN 5 showed very weak binding to fentanyl when compared to carfentanil (FIGS. 1A, 1C, and 1D).

TABLE 4
Complementary determining regions (CDRs)
of exemplary VHH sdAbs.

| sdAb | ID | CDR1 | ID | CDR2 | ID | CDR3 |
|---|---|---|---|---|---|---|
| JGFN1 | 1 | GDTFGITVIG | 6 | ARIYNTGTVK | 11 | AMGEADLT |
| | | | | | | HYDLW |
| JGFN2 | 2 | GFTLSTLDMR | 7 | STVSPDGNTY | 12 | ADISIAAH |
| | | | | | | EGGLPYW |
| JGFN3 | 3 | GFDFSRYDMG | 8 | SGIKSGGGPT | 13 | RDPDDHSG |
| | | | | V | | SYWGDYW |
| JGFN4 | 67 | GSTSRVNAMG | 9 | AAIDRSGATV | 14 | SGVLGSW |
| JGFN5 | 5 | GNTFSINSMA | 10 | AELHKTDRPG | 15 | ATGLWRDH |
| | | | | SVAAN | | EVW |

ID: SEQ ID NO

TABLE 5
Framework Regions 1 (FR1) and
2 (FR2) of exemplary VHH sdAbs.

| sdAb | ID | FR1 | ID | FR2 |
|---|---|---|---|---|
| JGFN1 | 16 | QVQLVESGGGLVQ | 21 | WHRQRAGQQQRELV |
| | | AGESLKLSCKAS | | |
| JGFN2 | 17 | QVQLVQSGGGLVQ | 22 | WFRQAPGKGFEWV |
| | | PGGSLRLSCAAS | | |
| JGFN3 | 18 | DVQLVESGGGMVQ | 23 | WVRQAPGKGPEWV |
| | | PGGSLRLSCVTS | | |
| JGFN4 | 19 | QVQLVESGGGLAQ | 24 | WYRQTPGKERELV |
| | | AGGSLQLSCAAS | | |
| JGFN5 | 20 | QVQLVESGGGLVQ | 25 | WYRQAPGKQRELV |
| | | AGESLNLSCTAS | | |

ID: SEQ ID NO

TABLE 6
Framework Regions 3 (FR3) and
4 (FR4) of exemplary VHH sdAbs.

| sdAb | ID | FR3 | ID | FR4 |
|---|---|---|---|---|
| JGFN1 | 26 | YADSVKGRFTLSSDSANDV | 31 | GPGTQVTVSS |
| | | VYLEMTDLKPEDTAVYYCH | | |
| JGFN2 | 27 | YSDSAKGRFAISRDVAKNT | 32 | GKGTPVTVSS |
| | | VNLQMSSLKPEDTAVYYCN | | |
| JGFN3 | 28 | YLDSVKGRFTVSRDNAKNT | 33 | GQGTLVTVSS |
| | | LYLQMNNLKPEDTARYYCA | | |
| JGFN4 | 68 | YSESVRGRFTISKNDAKNI | 34 | GLGTQVTVSS |
| | | VWLQMNNLTTEDTAVYYCR | | |
| JGFN5 | 30 | YADSVKGRFTIATDNSRDT | 35 | GQGTLVTVSS |
| | | MYLQMTNLKSADTATYFCY | | |

ID: SEQ ID NO

TABLE 7
Exemplary VHH sdAbs.

| sdAb | ID | Sequences |
|---|---|---|
| JGFN1 | 36 | QVQLVESGGGLVQAGESLKLSCKASGDTFGITVIG |
| | | WHRQRAGQQQRELVARIYNTGTVKYADSVKGRFTL |
| | | SSDSANDVVYLEMTDLKPEDTAVYYCHAMGEADLT |
| | | HYDLWGPGTQVTVSS |
| JGFN2 | 37 | QVQLVQSGGGLVQPGGSLRLSCAASGFTLSTLDMR |
| | | WFRQAPGKGFEWVSTVSPDGNTYYSDSAKGRFAIS |
| | | RDVAKNTVNLQMSSLKPEDTAVYYCNADISIAAHE |
| | | GGLPYWGKGTPVTVSS |
| JGFN3 | 38 | DVQLVESGGGMVQPGGSLRLSCVTSGFDFSRYDMG |
| | | WVRQAPGKGPEWVSGIKSGGGPTVYLDSVKGRFTV |
| | | SRDNAKNTLYLQMNNLKPEDTARYYCARDPDDHSG |
| | | SYWGDYWGQGTLVTVSS |
| JGFN4 | 39 | QVQLVESGGGLAQAGGSLQLSCAASGSTSRVNAMG |
| | | WYRQTPGKERELVAAIDRSGATVYSESVRGRFTIS |
| | | KNDAKNIVWLQMNNLTTEDTAVYYCRSGVLGSWGL |
| | | GTQVTVSS |
| JGFN5 | 40 | QVQLVESGGGLVQAGESLNLSCTASGNTFSINSMA |
| | | WYRQAPGKQRELVAELHKTDRPGSVAANYADSVKG |
| | | RFTIATDNSRDTMYLQMTNLKSADTATYFCYATGL |
| | | WRDHEVWGQGTLVTVSS |
| JGFN4 | 41 | QVQLVESGGGLAQAGGSLQLSCAASGSTSRVNAMG |
| N76D | | WYRQTPGKERELVAAIDRSGATVYSESVRGRFTIS |
| | | KNDAKDIVWLQMNNLTTEDTAVYYCRSGVLGSWGL |
| | | GTQVTVSS |
| JGFN4 | 42 | QVQLVESGGGLAQAGGSLQLSCAASGSTSRVNAMG |
| A74Y/N76D | | WYRQTPGKERELVAAIDRSGATVYSESVRGRFTIS |
| | | KNDYKDIVWLQMNNLTTEDTAVYYCRSGVLGSWGL |
| | | GTQVTVSS |
| JGFN4 | 43 | QVQLVESGGGLAQAGGSLQLSCAASGSTYRVNAMG |
| S29Y/N76D | | WYRQTPGKERELVAAIDRSGATVYSESVRGRFTIS |
| | | KNDAKDIVWLQMNNLTTEDTAVYYCRSGVLGSWGL |
| | | GTQVTVSS |
| JGFN4 | 44 | QVQLVESGGGLAQAGGSLQLSCAASGSTYRVNAMG |
| S29Y/A74Y/ | | WYRQTPGKERELVAAIDRSGATVYSESVRGRFTIS |
| N76D | | KNDYKDIVWLQMNNLTTEDTAVYYCRSGVLGSWGL |
| | | GTQVTVSS |

ID: SEQ ID NO

### JGFN4 Forms a Domain-Swapped Dimer to Bind Fentanyl

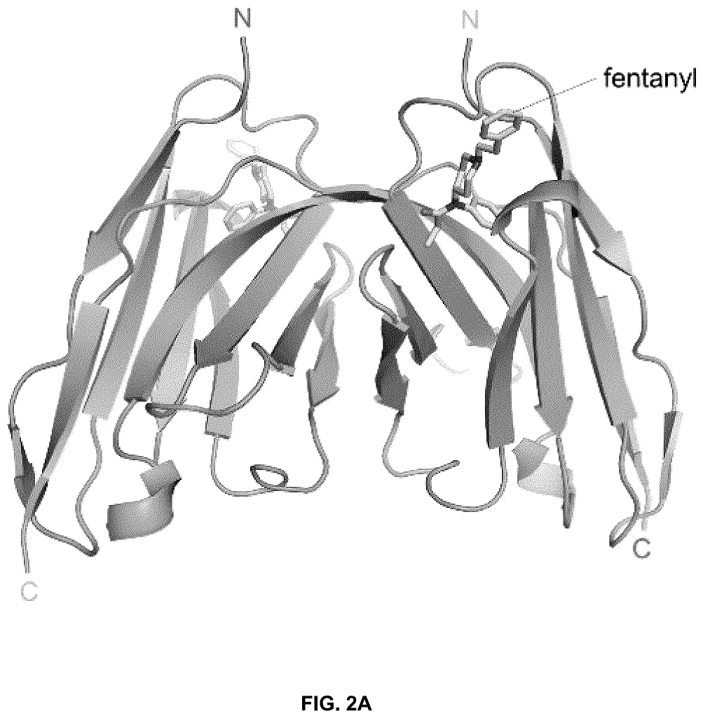

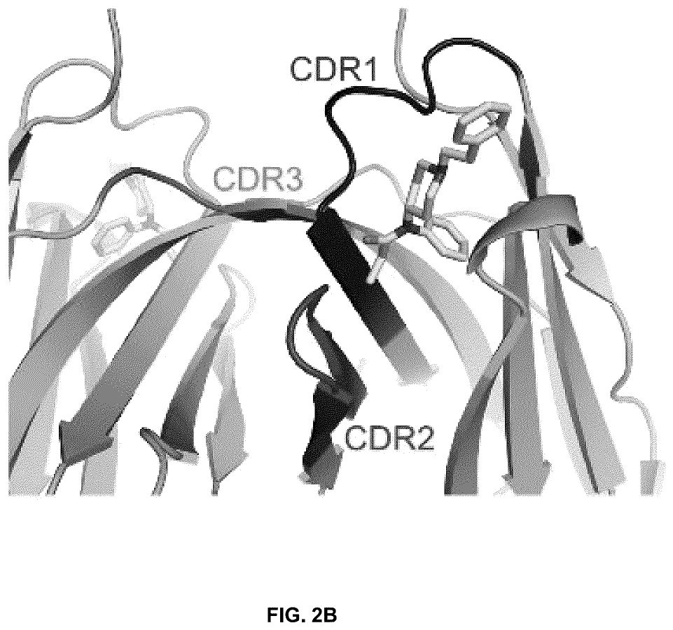

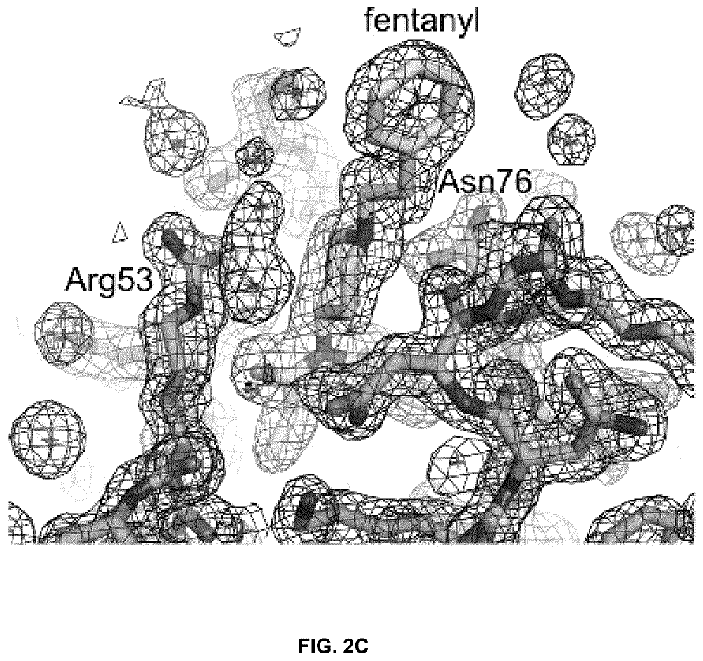

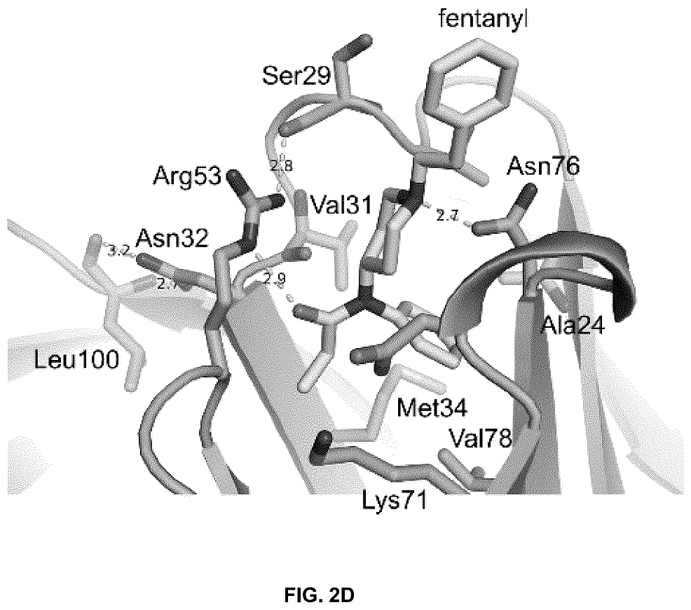

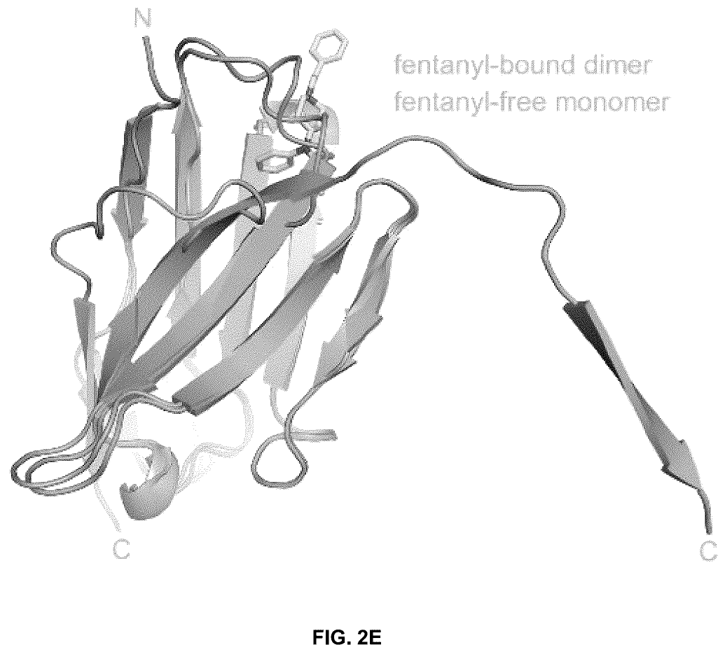

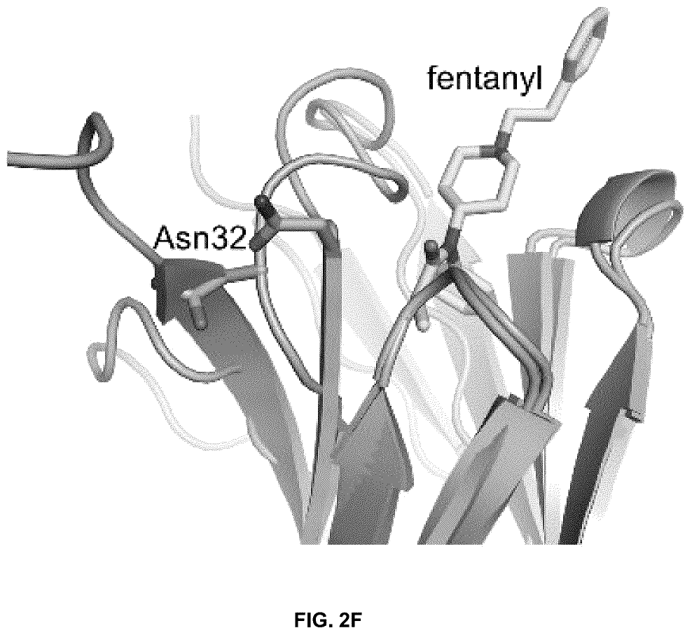

We next sought to determine the crystal structure of JGFN4 with and without fentanyl in order to understand how the VHH recognizes fentanyl. JGFN4 expressed in E. coli was purified as a mixture of monomers and non-covalent homodimers, separable by size-exclusion chromatography. Both forms were subjected to crystallization screening and yielded crystals in the presence of fentanyl. The structures were determined by molecular replacement phasing and refined to 1.68 and 1.55-Å resolution for crystals obtained with the monomeric and dimeric species, respectively (Table 3). Unexpectedly, both crystal structures showed a domain-swapped homodimer of JGFN4, with each protomer bound to one fentanyl molecule (FIG. 2A). Fentanyl was surrounded by CDR1, CDR2, and a juxtaposed loop between CDR2 and CDR3, which formed a turn of a helix (FIG. 2B). The CDR3, which typically forms a long loop responsible for making key interactions with the antigen by VHHs³², instead crossed over to the other protomer (trans conformation) to mediate a reciprocal swapping of the C-terminal β-strand of JGFN4.

The unique characteristics of the fentanyl binding pocket were next investigated. The N-phenylpropanamide moiety of fentanyl is inserted into a deep pocket formed between the two layers of β-sheets that form the core of the VHH. Hydrophobic contacts are made with Ala24, Met34, Lys71, Val78, and Arg53 side chains and van der Waals contacts with the main chain atoms of Val31 and Asn32 (FIGS. 2C and 2D). Arg53 from CDR2 also donates a hydrogen bond to the carbonyl oxygen atom of fentanyl while hydrogen-bonded to the Ser29 carbonyl group from CDR1 to help shape the pocket. Asn76 accepts a hydrogen bond from the tertiary amine nitrogen atom of the piperidine moiety. The piperidine ring is also stabilized by extensive van der Waals contacts with protein residues from CDR1 and the short helix between CDR2 and CDR3 mentioned above. The phenylethyl moiety N-linked to the piperidine was pointed away from the protein and exposed to the solvent, where its conformation was crystal form-dependent.

We obtained crystals of isolated JGFN4 monomers in the absence of fentanyl and determined the structure at 1.76-Å resolution. The structure shows monomeric JGFN4 without domain swapping—the CDR3 forms a turn in this structure (cis conformation), allowing the C-terminal β-strand to complete the VHH fold intramolecularly (FIGS. 2E and 2F). The CDR1 loop partially collapses into the unoccupied fentanyl-binding pocket. Patchy electron density and high B-factors in the refined model suggest that the CDR1 loop is highly flexible in the absence of fentanyl. These observations suggest that JGFN4 preferentially binds fentanyl in the domain-swapped dimer and that crystallization in the presence of fentanyl likely promoted VHH dimerization. A superposition of the fentanyl-free JGFN4 monomer with the fentanyl-bound JGFN4 dimer highlights distinct conformations of CDR3 and neighboring structural elements. Of particular interest is Asn32 from CDR1, whose side chain undergoes a −4.5 Å shift upon the transition of CDR3 from the cis to trans conformations (FIG. 2F). In the trans conformation, Leu100 from the straightened CDR3 formed bidentate hydrogen bonds with the side chain of Asn32, which accompanies a −3 Å shift of the Ca position to shape the fentanyl-binding pocket.

### N76D Mutation Allows JGFN4 Monomers to Bind Fentanyl

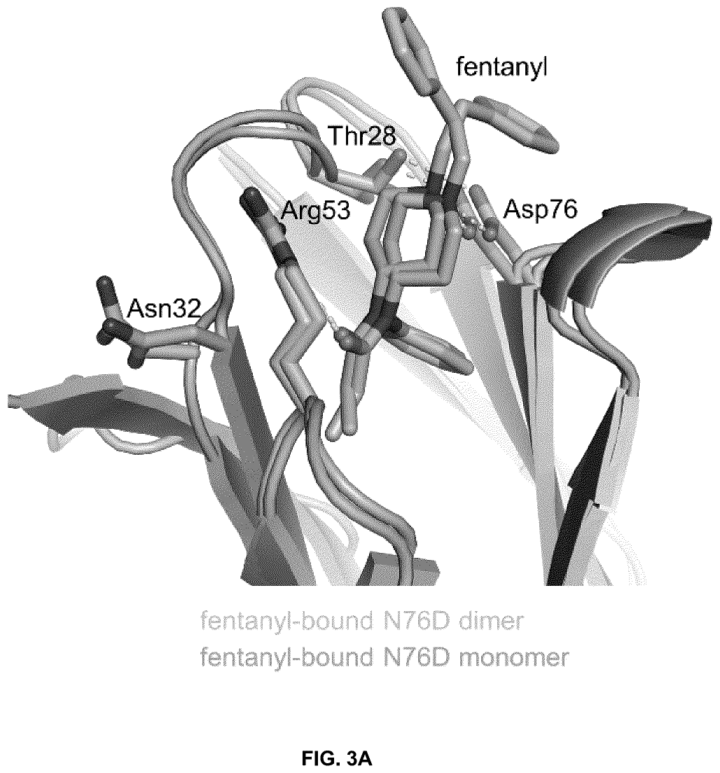

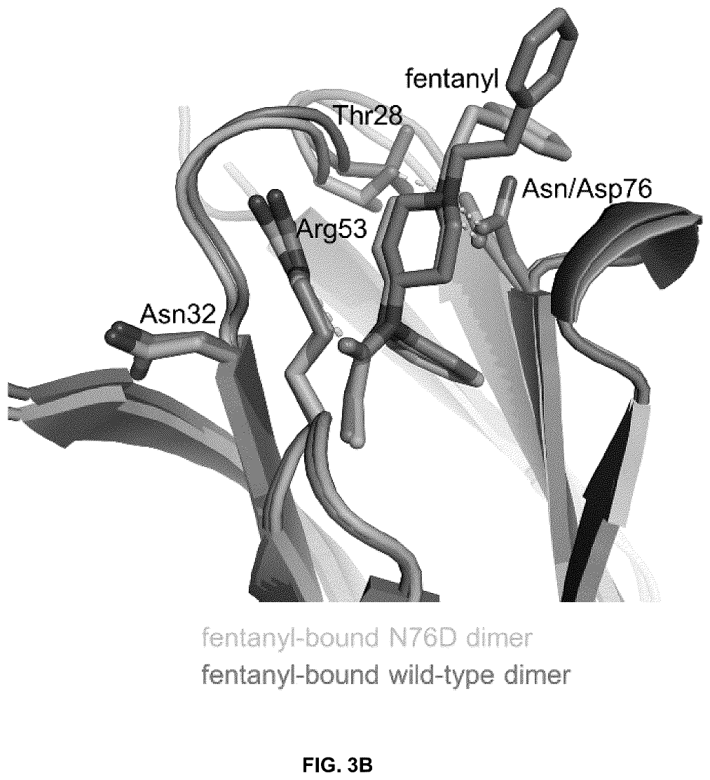

Our structural data suggest that hydrogen bonding between Asn76 and the piperidine moiety of fentanyl is a key interaction for VHH engagement. To obtain further insights into the mechanism of fentanyl binding by JGFN4 and to improve the binding affinity, we generated a point mutant N76D of JGFN4 to strengthen its polar interaction with the piperidine moiety potentially. As observed for wild-type JGFN4, we obtained monomeric and dimeric forms of JGFN4 N76D when it was expressed in E. coli for structural studies. Both species yielded crystals in the presence of fentanyl, and the structures were determined at 1.60 and 1.85-Å resolution. As expected, crystals obtained with the dimeric form showed a domain-swapped dimer with the trans conformation of CDR3 and fentanyl bound to each protomer (FIG. 3). Surprisingly, however, the structure for the crystals grown with monomeric JGFN4 N76D was found to be a monomer with bound fentanyl. Thus, it appears that the N76D mutation alleviates the need for JGFN4 dimerization via domain swapping in fentanyl-binding. The substituted Asp76 side chain forms a hydrogen bond not only with the tertiary amine nitrogen of the piperidine moiety but also with the side chain of Thr28 from CDR1 (FIG. 3A). Asn76 and Thr28 are ˜1.0 Å farther apart in the wild-type structure. This shift also brings the Thr28 side chain closer by ˜1.0 Å to the piperidine ring of fentanyl. Given the flexibility of CDR1 as observed in the fentanyl-free structure, stabilization of CDR1 in the fentanyl-bound conformation, and an enhanced van der Waals contact provided by the N76D mutation likely account for its improved affinity for fentanyl.

While a structural comparison between the fentanyl-bound JGFN4 dimer and fentanyl-free JGFN4 monomer was instructive, having the monomeric and dimeric JGFN4 N76D structures both in complex with fentanyl allows for a more direct comparison to understand how the JGFN4 dimerization benefits fentanyl binding. A superposition of the two structures shows that the N-phenylpropanamide moiety of fentanyl is more tightly surrounded by protein residues in the dimeric structure (FIG. 3B). Asn32 is positioned closer to fentanyl due to its interaction with Leu100 in the extended trans conformation (e.g., Asn32 Ca to the carbonyl oxygen atom of fentanyl is 1.2 Å closer) in the dimeric structure. We reason that the flexibility of glycine residues in the unique CDR3 sequence of JGFN4 (⁹⁸GVLG¹⁰¹) promotes switching to the trans conformation, which helps to shape a tighter binding pocket for fentanyl through its interaction with CDR1 residues including Asn32.

### Mutations Increase the Affinity for Fentanyl in Dimerized JGFN4

Based on the crystallization studies indicating that wild type JGFN4 bound fentanyl as a dimer, we expressed JGFN4 as two tandem repeats connected by a (Gly₄S)₄ linker in an attempt to force dimerization. The binding affinity of dimerized wild type JGFN4 for immobilized F₁ fentanyl hapten was determined to be 33 nM by biolayer interferometry (BLI). Rational mutagenesis using the structural data was next performed to improve binding. Ala74 was mutated to Tyr or Trp to increase possible pi-stacking with the phenyl ring of fentanyl while Asn76 was mutated to Asp or His to increase hydrogen bond interactions and coordination of tertiary amine of fentanyl (Table 8). JGFN4 N76D showed an approximately 3-fold increase in binding compared to wild type while the A74Y demonstrated a modest increase in affinity. When combined the A74Y/N76D double mutant had a K_{D} of 8.65 nM slightly better than JGFN4 N76D. An additional mutation S29Y was investigated, but did not show a significant increase in affinity as either a double or triple mutant further highlighting the importance of N76D in fentanyl binding.

BLI is a kinetic method of affinity determination using immobilized F1 hapten, whereas competitive ELISA and differential scanning fluorimetry (DSF) use free fentanyl. Therefore, BLI is likely to over-estimate the affinity of dimeric proteins due to the reduced off-rate when both binding sites are bound to the sensor, which does not accurately reflect the behavior of free fentanyl in solution. To verify the binding of JGFN4 and the mutants to free drug, competitive ELISA and DSF were employed. WT JGFN4 showed no binding to fentanyl by competitive ELISA and a very low affinity by DSF of 155 μM while N76D demonstrated an increase in affinity relative to WT by both methods. Next, the double mutants of N76D combined with A74Y and S29Y were tested. These mutants showed improved binding by BLI and DSF, but similar affinity to N76D alone by competitive ELISA.

TABLE 8

| JGFN4 VHH | BLI KD | Competitive ELISA | DSF |
|---|---|---|---|
| Mutant | (nM) | IC50 (nM) | KD (nM) |
| WT | 33.69 | No Binding | ~154,953 |
| A74Y | 21.72 | 58,600 | ~325,750 |
| A74W | 31.31 | 65,100 | Not Tested |
| N76D | 11.03 | 35,200 | ~79,391 |
| N76H | 17.90 | No Binding | Not Tested |
| A74Y/N76D | 8.65* | 42,700 | ~22,603 |
| T28D/N76Y | NDB | Not Tested | Not Tested |
| S29Y/N76D | 7.66* | 55,100 | ~32,943 |
| S29Y/A74Y/N76D | 7.14 | Not Tested | Not Tested |

*Average of two runs

### REFERENCES

ADDIN EN.REFLIST 1. Compton, P. The United States opioid crisis: Big pharma alone is not to blame.”. Prev Med 177, 107777 (2023).
2. Sullivan, P. S., Bradley, H. M., Rio, C. D. & Rosenberg, E. S. The Geography of Opioid Use Disorder: A Data Triangulation Approach. Infect Dis Clin North Am 34, 451-464 (2020).
3. Skolnick, P. Treatment of overdose in the synthetic opioid era. Pharmacol Ther 233, 108019 (2022).
4. Cano, M., Timmons, P., Hooten, M. & Sweeney, K. Drug supply measures and drug overdose mortality in the era of fentanyl and stimulants. Drug Alcohol Depend Rep 9, 100197 (2023).
5. Kukanich, B. & Clark, T. P. The history and pharmacology of fentanyl: relevance to a novel, long-acting transdermal fentanyl solution newly approved for use in dogs. J Vet Pharmacol Ther 35 Suppl 2, 3-19 (2012).
6. Burns, S. M., Cunningham, C. W. & Mercer, S. L. DARK Classics in Chemical Neuroscience: Fentanyl. ACS Chem Neurosci 9, 2428-2437 (2018).
7. Albores-Garcia, D. & Cruz, S. L. Fentanyl and other New Psychoactive Synthetic Opioids. Challenges to Prevention and Treatment. Rev Invest Clin 75, 93-104 (2023).
8. Hartmann, G. E. & Sethi, R. The Role of Non-Pharmaceutical Fentanyl-Contaminated Counterfeit Oxycodone in Increasing Opioid Overdoses: A Commentary Review. Prim Care Companion CNS Disord 25(2023).
9. Santelices, C., Matsumoto, A., Boulad, M. & Stopka, T. J. Evaluating Technologies to Identify Fentanyl and Adulterants in Street Drug Paraphernalia: Qualitative Perspectives of Service Providers and Their Clientele. Subst Use Misuse 58, 1528-1535 (2023).
10. Jordan, M. R., Lopez, R. A. & Morrisonponce, D. Asystole. in StatPearls (Treasure Island (FL), 2023).
11. Salinsky, L. M., et al. mu-opioid receptor agonists and psychedelics: pharmacological opportunities and challenges. Front Pharmacol 14, 1239159 (2023).
12. Kang, Y., et al. Naloxone's dose-dependent displacement of [(11)C]carfentanil and duration of receptor occupancy in the rat brain. Sci Rep 12, 6429 (2022).
13. Luba, R., Martinez, S., Jones, J., Pravetoni, M. & Corner, S. D. Immunotherapeutic strategies for treating opioid use disorder and overdose. Expert Opin Investig Drugs 32, 77-87 (2023).
14. France, C. P., et al. Countermeasures for Preventing and Treating Opioid Overdose. Clin Pharmacol Ther 109, 578-590 (2021).
15. Pravetoni, M. Biologics to treat substance use disorders: Current status and new directions. Hum Vaccin Immunother 12, 3005-3019 (2016).
16. Powers, N., et al. Self-Adjuvanting TLR7/8 Agonist and Fentanyl Hapten Co-Conjugate Achieves Enhanced Protection against Fentanyl Challenge. Bioconjug Chem 34, 1811-1821 (2023).
17. Raleigh, M. D., et al. Pharmacological mechanisms underlying the efficacy of antibodies generated by a vaccine to treat oxycodone use disorder. Neuropharmacology 195, 108653 (2021).
18. Park, H., et al. Improvements on a chemically contiguous hapten for a vaccine to address fentanyl-contaminated heroin. Bioorg Med Chem 41, 116225 (2021).
19. Haile, C. N., et al. An Immunconjugate Vaccine Alters Distribution and Reduces the Antinociceptive, Behavioral and Physiological Effects of Fentanyl in Male and Female Rats. Pharmaceutics 14(2022).
20. Tenney, R. D., et al. Vaccine blunts fentanyl potency in male rhesus monkeys. Neuropharmacology 158, 107730 (2019).
21. Townsend, E. A., et al. Evaluation of a Dual Fentanyl/Heroin Vaccine on the Antinociceptive and Reinforcing Effects of a Fentanyl/Heroin Mixture in Male and Female Rats. ACS Chem Neurosci 11, 1300-1310 (2020).
22. Pravetoni, M. & Corner, S. D. Development of vaccines to treat opioid use disorders and reduce incidence of overdose. Neuropharmacology 158, 107662 (2019).
23. Smith, L. C., et al. Monoclonal Antibodies for Combating Synthetic Opioid Intoxication. J Am Chem Soc 141, 10489-10503 (2019).
24. Eubanks, L. M., et al. An Engineered Human-Antibody Fragment with Fentanyl Pan-Specificity That Reverses Carfentanil-Induced Respiratory Depression. ACS Chem Neurosci 14, 2849-2856 (2023).
25. Rodarte, J. V., et al. Structures of drug-specific monoclonal antibodies bound to opioids and nicotine reveal a common mode of binding. Structure 31, 20-32 e25 (2023).
26. Hicks, D., et al. Advancing humanized monoclonal antibody for counteracting fentanyl toxicity towards clinical development. Hum Vaccin Immunother 18, 2122507 (2022).
27. Bathula, N. V., Bommadevara, H. & Hayes, J. M. Nanobodies: The Future of Antibody-Based Immune Therapeutics. Cancer Biother Radiopharm 36, 109-122 (2021).
28. Steeland, S., Vandenbroucke, R. E. & Libert, C. Nanobodies as therapeutics: big opportunities for small antibodies. Drug Discov Today 21, 1076-1113 (2016).
29. Modhiran, N., et al. A nanobody recognizes a unique conserved epitope and potently neutralizes SARS-CoV-2 omicron variants. iScience 26, 107085 (2023).
30. Ye, G., et al. The development of Nanosota-1 as anti-SARS-CoV-2 nanobody drug candidates. Elife 10(2021).
31. Ackaert, C., et al. Immunogenicity Risk Profile of Nanobodies. Front Immunol 12, 632687 (2021).
32. Bannas, P., Hambach, J. & Koch-Nolte, F. Nanobodies and Nanobody-Based Human Heavy Chain Antibodies As Antitumor Therapeutics. Front Immunol 8, 1603 (2017).
33. Robinson, C., et al. Therapeutic and Prophylactic Vaccines to Counteract Fentanyl Use Disorders and Toxicity. J Med Chem 63, 14647-14667 (2020).
34. Baehr, C., et al. Monoclonal Antibodies Counteract Opioid-Induced Behavioral and Toxic Effects in Mice and Rats. J Pharmacol Exp Ther 375, 469-477 (2020).
35. Baehr, C., et al. Preclinical Efficacy and Selectivity of Vaccines Targeting Fentanyl, Alfentanil, Sufentanil, and Acetylfentanyl in Rats. ACS Omega 7, 16584-16592 (2022).
36. Ye, G., et al. The Development of a Novel Nanobody Therapeutic for SARS-CoV-2. bioRxiv, 2020.2011.2017.386532 (2020).
37. Hintz, H. M., Cowan, A. E., Shapovalova, M. & LeBeau, A. M. Development of a Cross-Reactive Monoclonal Antibody for Detecting the Tumor Stroma. Bioconjug Chem 30, 1466-1476 (2019).
38. Glumac, P. M., et al. Exploitation of CD133 for the Targeted Imaging of Lethal Prostate Cancer. Clin Cancer Res 26, 1054-1064 (2020).
39. Kabsch, W. Xds. Acta Crystallogr D Biol Crystallogr 66, 125-132 (2010).
40. McCoy, A. J., et al. Phaser crystallographic software. J Appl Crystallogr 40, 658-674 (2007).
41. Gulati, S., et al. Targeting G protein-coupled receptor signaling at the G protein level with a selective nanobody inhibitor. Nat Commun 9, 1996 (2018).
42. Emsley, P., Lohkamp, B., Scott, W. G. & Cowtan, K. Features and development of Coot. Acta Crystallogr D Biol Crystallogr 66, 486-501 (2010).
43. Adams, P. D., et al. PHENIX: a comprehensive Python-based system for macromolecular structure solution. Acta Crystallogr D Biol Crystallogr 66, 213-221 (2010).
44. Kirley T L, Norman A B, Wetzel H N. A novel differential scanning fluorimetry analysis of a humanized anti-cocaine mAb and its ligand binding characteristics. J Immunol Methods. 2020 January; 476: 112676.

What is claimed is:

1. An isolated anti-opioid construct comprising a single-domain antibody (sdAb) moiety specifically recognizing an opioid selected from the group consisting of fentanyl and carfentanil, wherein the sdAb moiety comprises a CDR1, a CDR2, and a CDR3, wherein:
the CDR1 comprises an amino acid sequence of GDTFGITVIG (SEQ ID NO: 1), GFTLSTLDMR (SEQ ID NO: 2), GFDFSRYDMG (SEQ ID NO: 3), GSX¹X²RVNAMG (SEQ ID NO: 4) wherein X¹ is T or D and X² is S or Y, GNTFSINSMA (SEQ ID NO: 5), or a variant of any of the foregoing comprising up to 3 amino acid substitutions;
the CDR2 comprises an amino acid sequence of ARIYNTGTVK (SEQ ID NO: 6), STVSPDGNTY (SEQ ID NO: 7), SGIKSGGGPTV (SEQ ID NO: 8), AAIDRSGATV (SEQ ID NO: 9), AELHKTDRPGSVAAN (SEQ ID NO: 10), or a variant of any of the foregoing comprising up to 3 amino acid substitutions; and
the CDR3 comprises an amino acid sequence of AMGEADLTHYDLW (SEQ ID NO: 11), ADISIAAHEGGLPYW (SEQ ID NO: 12), DPDDHSGSYWGDYW (SEQ ID NO: 13), SGVLGSW (SEQ ID NO: 14), ATGLWRDHEVW (SEQ ID NO: 15), or a variant of any of the foregoing comprising up to 3 amino acid substitutions.

2. The isolated anti-opioid construct of claim 1, wherein:
the CDR1 comprises an amino acid sequence of GDTFGITVIG (SEQ ID NO: 1), GFTLSTLDMR (SEQ ID NO: 2), GFDFSRYDMG (SEQ ID NO: 3), or GSX¹X²RVNAMG (SEQ ID NO: 4) wherein X¹ is T or D and X² is S or Y, GNTFSINSMA (SEQ ID NO: 5), or a variant of any of the foregoing comprising up to 1 amino acid substitution;
the CDR2 comprises an amino acid sequence of ARIYNTGTVK (SEQ ID NO: 6), STVSPDGNTY (SEQ ID NO: 7), SGIKSGGGPTV (SEQ ID NO: 8), AAIDRSGATV (SEQ ID NO: 9), AELHKTDRPGSVAAN (SEQ ID NO: 10), or a variant of any of the foregoing comprising up to 1 amino acid substitution; and
the CDR3 comprises an amino acid sequence of AMGEADLTHYDLW (SEQ ID NO: 11), ADISIAAHEGGLPYW (SEQ ID NO: 12), DPDDHSGSYWGDYW (SEQ ID NO: 13), SGVLGSW (SEQ ID NO: 14), ATGLWRDHEVW (SEQ ID NO: 15), or a variant of any of the foregoing comprising up to 1 amino acid substitution.

3. The isolated anti-opioid construct of claim 1, wherein:
the CDR1 comprises an amino acid sequence of GDTFGITVIG (SEQ ID NO: 1), GFTLSTLDMR (SEQ ID NO: 2), GFDFSRYDMG (SEQ ID NO: 3), or GSX¹X²RVNAMG (SEQ ID NO: 4) wherein X¹ is T or D and X² is S or Y, or GNTFSINSMA (SEQ ID NO: 5);
the CDR2 comprises an amino acid sequence of ARIYNTGTVK (SEQ ID NO: 6), STVSPDGNTY (SEQ ID NO: 7), SGIKSGGGPTV (SEQ ID NO: 8), AAIDRSGATV (SEQ ID NO: 9), or AELHKTDRPGSVAAN (SEQ ID NO: 10); and
the CDR3 comprises an amino acid sequence of AMGEADLTHYDLW (SEQ ID NO: 11), ADISIAAHEGGLPYW (SEQ ID NO: 12), DPDDHSGSYWGDYW (SEQ ID NO: 13), SGVLGSW (SEQ ID NO: 14), or ATGLWRDHEVW (SEQ ID NO: 15).

4. The isolated anti-opioid construct of claim 1, wherein:
the CDR1 comprises an amino acid sequence of GDTFGITVIG (SEQ ID NO: 1) or a variant thereof comprising up to 3 amino acid substitutions, the CDR2 comprises an amino acid sequence of ARIYNTGTVK (SEQ ID NO: 6) or a variant thereof comprising up to 3 amino acid substitutions, and the CDR3 comprises an amino acid sequence of AMGEADLTHYDLW (SEQ ID NO: 11) or a variant thereof comprising up to 3 amino acid substitutions;
the CDR1 comprises an amino acid sequence of GFTLSTLDMR (SEQ ID NO: 2) or a variant thereof comprising up to 3 amino acid substitutions, the CDR2 comprises an amino acid sequence of STVSPDGNTY (SEQ ID NO: 7) or a variant thereof comprising up to 3 amino acid substitutions, and the CDR3 comprises an amino acid sequence of ADISIAAHEGGLPYW (SEQ ID NO: 12) or a variant thereof comprising up to 3 amino acid substitutions;
the CDR1 comprises an amino acid sequence of GFDFSRYDMG (SEQ ID NO: 3) or a variant thereof comprising up to 3 amino acid substitutions, the CDR2 comprises an amino acid sequence of SGIKSGGGPTV (SEQ ID NO: 8) or a variant thereof comprising up to 3 amino acid substitutions, and the CDR3 comprises an amino acid sequence of DPDDHSGSYWGDYW (SEQ ID NO: 13) or a variant thereof comprising up to 3 amino acid substitutions;
the CDR1 comprises an amino acid sequence of GSX¹X²RVNAMG (SEQ ID NO: 4) wherein X¹ is T or D and X² is S or Y or a variant thereof comprising up to 3 amino acid substitutions, the CDR2 comprises an amino acid sequence of AAIDRSGATV (SEQ ID NO: 9) or a variant thereof comprising up to 3 amino acid substitutions, and the CDR3 comprises an amino acid sequence of SGVLGSW (SEQ ID NO: 14) or a variant thereof comprising up to 3 amino acid substitutions; or
the CDR1 comprises an amino acid sequence of GNTFSINSMA (SEQ ID NO: 5) or a variant thereof comprising up to 3 amino acid substitutions, the CDR2 comprises an amino acid sequence of AELHKTDRPGSVAAN (SEQ ID NO: 10) or a variant thereof comprising up to 3 amino acid substitutions, and the CDR3 comprises an amino acid sequence of ATGLWRDHEVW (SEQ ID NO: 15) or a variant thereof comprising up to 3 amino acid substitutions.

5. The isolated anti-opioid construct of claim 1, wherein:
the CDR1 comprises an amino acid sequence of GDTFGITVIG (SEQ ID NO: 1) or a variant thereof comprising up to 1 amino acid substitution, the CDR2 comprises an amino acid sequence of ARIYNTGTVK (SEQ ID NO: 6) or a variant thereof comprising up to 1 amino acid substitution, and the CDR3 comprises an amino acid sequence of AMGEADLTHYDLW (SEQ ID NO: 11) or a variant thereof comprising up to 1 amino acid substitution;
the CDR1 comprises an amino acid sequence of GFTLSTLDMR (SEQ ID NO: 2) or a variant thereof comprising up to 1 amino acid substitution, the CDR2 comprises an amino acid sequence of STVSPDGNTY (SEQ ID NO: 7) or a variant thereof comprising up to 1 amino acid substitution, and the CDR3 comprises an amino acid sequence of ADISIAAHEGGLPYW (SEQ ID NO: 12) or a variant thereof comprising up to 1 amino acid substitution;
the CDR1 comprises an amino acid sequence of GFDFSRYDMG (SEQ ID NO: 3) or a variant thereof comprising up to 1 amino acid substitution, the CDR2 comprises an amino acid sequence of SGIKSGGGPTV (SEQ ID NO: 8) or a variant thereof comprising up to 1 amino acid substitution, and the CDR3 comprises an amino acid sequence of DPDDHSGSYWGDYW (SEQ ID NO: 13) or a variant thereof comprising up to 1 amino acid substitution;
the CDR1 comprises an amino acid sequence of GSX¹X²RVNAMG (SEQ ID NO: 4) wherein X¹ is T or D and X² is S or Y or a variant thereof comprising up to 1 amino acid substitution, the CDR2 comprises an amino acid sequence of AAIDRSGATV (SEQ ID NO: 9) or a variant thereof comprising up to 1 amino acid substitution, and the CDR3 comprises an amino acid sequence of SGVLGSW (SEQ ID NO: 14) or a variant thereof comprising up to 1 amino acid substitution; or
the CDR1 comprises an amino acid sequence of GNTFSINSMA (SEQ ID NO: 5) or a variant thereof comprising up to 1 amino acid substitution, the CDR2 comprises an amino acid sequence of AELHKTDRPGSVAAN (SEQ ID NO: 10) or a variant thereof comprising up to 1 amino acid substitution, and the CDR3 comprises an amino acid sequence of ATGLWRDHEVW (SEQ ID NO: 15) or a variant thereof comprising up to 1 amino acid substitution.

6. The isolated anti-opioid construct of claim 5, wherein the sdAb moiety comprises an amino acid sequence having at least 95% sequence identity to an sdAb sequence selected from the group consisting of SEQ ID NOs:36-44.

7. The isolated anti-opioid construct of claim 1, wherein:
the CDR1 comprises an amino acid sequence of GDTFGITVIG (SEQ ID NO: 1), the CDR2 comprises an amino acid sequence of ARIYNTGTVK (SEQ ID NO: 6), and the CDR3 comprises an amino acid sequence of AMGEADLTHYDLW (SEQ ID NO: 11);
the CDR1 comprises an amino acid sequence of GFTLSTLDMR (SEQ ID NO: 2), the CDR2 comprises an amino acid sequence of STVSPDGNTY (SEQ ID NO: 7), and the CDR3 comprises an amino acid sequence of ADISIAAHEGGLPYW (SEQ ID NO: 12);
the CDR1 comprises an amino acid sequence of GFDFSRYDMG (SEQ ID NO: 3), the CDR2 comprises an amino acid sequence of SGIKSGGGPTV (SEQ ID NO: 8), and the CDR3 comprises an amino acid sequence of DPDDHSGSYWGDYW (SEQ ID NO: 13);
the CDR1 comprises an amino acid sequence of GSX¹X²RVNAMG (SEQ ID NO: 4) wherein X¹ is T or D and X² is S or Y, the CDR2 comprises an amino acid sequence of AAIDRSGATV (SEQ ID NO: 9), and the CDR3 comprises an amino acid sequence of SGVLGSW (SEQ ID NO: 14); or
the CDR1 comprises an amino acid sequence of GNTFSINSMA (SEQ ID NO: 5), the CDR2 comprises an amino acid sequence of AELHKTDRPGSVAAN (SEQ ID NO: 10), and the CDR3 comprises an amino acid sequence of ATGLWRDHEVW (SEQ ID NO: 15).

8. The isolated anti-opioid construct of claim 7, wherein the sdAb moiety comprises an amino acid sequence having at least 95% sequence identity to an sdAb sequence selected from the group consisting of SEQ ID NOs:36-44.

9. The isolated anti-opioid construct of claim 1, wherein the sdAb moiety comprises an FR1, an FR2, an FR3, and an FR4, wherein:
the FR1 comprises an amino acid sequence of QVQLVESGGGLVQAGESLKLSCKAS (SEQ ID NO: 16), QVQLVQSGGGLVQPGGSLRLSCAAS (SEQ ID NO: 17), DVQLVESGGGMVQPGGSLRLSCVTS (SEQ ID NO: 18), QVQLVESGGGLAQAGGSLQLSCAAS (SEQ ID NO: 19), QVQLVESGGGLVQAGESLNLSCTAS (SEQ ID NO: 20), or a variant of any of the foregoing comprising up to 3 amino acid substitutions;
the FR2 comprises an amino acid sequence of WHRQRAGQQQRELV (SEQ ID NO: 21), WFRQAPGKGFEWV (SEQ ID NO: 22), WVRQAPGKGPEWV (SEQ ID NO: 23), WYRQTPGKERELV (SEQ ID NO: 24), WYRQAPGKQRELV (SEQ ID NO: 25), or a variant of any of the foregoing comprising up to 3 amino acid substitutions;
the FR3 comprises an amino acid sequence of YADSVKGRFTLSSDSANDVVYLEMTDLKPEDTAVYYCH (SEQ ID NO: 26), YSDSAKGRFAISRDVAKNTVNLQMSSLKPEDTAVYYCN (SEQ ID NO: 27), YLDSVKGRFTVSRDNAKNTLYLQMNNLKPEDTARYYCA (SEQ ID NO: 28), YSESVRGRFTISKNDX³KX⁴IVWLQMNNLTTEDTAVYYCR (SEQ ID NO: 29) wherein X³ is A, Y, or W and X⁴ is N, D, H, or Y, YADSVKGRFTIATDNSRDTMYLQMTNLKSADTATYFCY (SEQ ID NO: 30), or a variant of any of the foregoing comprising up to 3 amino acid substitutions; and/or
the FR4 comprises an amino acid sequence of GPGTQVTVSS (SEQ ID NO: 31), GKGTPVTVSS (SEQ ID NO: 32), GQGTLVTVSS (SEQ ID NO: 33), GLGTQVTVSS (SEQ ID NO: 34), GQGTLVTVSS (SEQ ID NO: 35), or a variant of any of the foregoing comprising up to 3 amino acid substitutions.

10. The isolated anti-opioid construct of claim 1, wherein the sdAb moiety comprises an FR1, an FR2, an FR3, and an FR4, wherein:
the FR1 comprises an amino acid sequence of QVQLVESGGGLVQAGESLKLSCKAS (SEQ ID NO: 16) or a variant thereof comprising up to 3 amino acid substitutions, the FR2 comprises an amino acid sequence of WHRQRAGQQQRELV (SEQ ID NO: 21) or a variant thereof comprising up to 3 amino acid substitutions, the FR3 comprises an amino acid sequence of YADSVKGRFTLSSDSANDVVYLEMTDLKPEDTAVYYCH (SEQ ID NO: 26) or a variant thereof comprising up to 3 amino acid substitutions, and the FR4 comprises an amino acid sequence of GPGTQVTVSS (SEQ ID NO: 31) or a variant thereof comprising up to 3 amino acid substitutions;
the FR1 comprises an amino acid sequence of QVQLVQSGGGLVQPGGSLRLSCAAS (SEQ ID NO: 17) or a variant thereof comprising up to 3 amino acid substitutions, the FR2 comprises an amino acid sequence of WFRQAPGKGFEWV (SEQ ID NO: 22) or a variant thereof comprising up to 3 amino acid substitutions, the FR3 comprises an amino acid sequence of YSDSAKGRFAISRDVAKNTVNLQMSSLKPEDTAVYYCN (SEQ ID NO: 27) or a variant thereof comprising up to 3 amino acid substitutions, and the FR4 comprises an amino acid sequence of GKGTPVTVSS (SEQ ID NO: 32) or a variant thereof comprising up to 3 amino acid substitutions;
the FR1 comprises an amino acid sequence of DVQLVESGGGMVQPGGSLRLSCVTS (SEQ ID NO: 18) or a variant thereof comprising up to 3 amino acid substitutions, the FR2 comprises an amino acid sequence of WVRQAPGKGPEWV (SEQ ID NO: 23) or a variant thereof comprising up to 3 amino acid substitutions, the FR3 comprises an amino acid sequence of YLDSVKGRFTVSRDNAKNTLYLQMNNLKPEDTARYYCA (SEQ ID NO: 28) or a variant thereof comprising up to 3 amino acid substitutions, and the FR4 comprises an amino acid sequence of GQGTLVTVSS (SEQ ID NO: 33) or a variant thereof comprising up to 3 amino acid substitutions;
the FR1 comprises an amino acid sequence of QVQLVESGGGLAQAGGSLQLSCAAS (SEQ ID NO: 19) or a variant thereof comprising up to 3 amino acid substitutions, the FR2 comprises an amino acid sequence of WYRQTPGKERELV (SEQ ID NO: 24) or a variant thereof comprising up to 3 amino acid substitutions, the FR3 comprises an amino acid sequence of YSESVRGRFTISKNDX³KX⁴IVWLQMNNLTTEDTAVYYCR (SEQ ID NO: 29) wherein X³ is A, Y, or W and X⁴ is N, D, H, or Y or a variant thereof comprising up to 3 amino acid substitutions, and the FR4 comprises an amino acid sequence of GLGTQVTVSS (SEQ ID NO: 34) or a variant thereof comprising up to 3 amino acid substitutions; or
the FR1 comprises an amino acid sequence of QVQLVESGGGLVQAGESLNLSCTAS (SEQ ID NO: 20) or a variant thereof comprising up to 3 amino acid substitutions, the FR2 comprises an amino acid sequence of WYRQAPGKQRELV (SEQ ID NO: 25) or a variant thereof comprising up to 3 amino acid substitutions, the FR3 comprises an amino acid sequence of YADSVKGRFTIATDNSRDTMYLQMTNLKSADTATYFCY (SEQ ID NO: 30) or a variant thereof comprising up to 3 amino acid substitutions, and the FR4 comprises an amino acid sequence of GQGTLVTVSS (SEQ ID NO: 35) or a variant thereof comprising up to 3 amino acid substitutions.

11. The isolated anti-opioid construct of claim 1, wherein the sdAb moiety comprises an FR1, an FR2, an FR3, and an FR4, wherein:
the CDR1 comprises an amino acid sequence of GDTFGITVIG (SEQ ID NO: 1), the CDR2 comprises an amino acid sequence of ARIYNTGTVK (SEQ ID NO: 6), the CDR3 comprises an amino acid sequence of AMGEADLTHYDLW (SEQ ID NO: 11), the FR1 comprises an amino acid sequence of QVQLVESGGGLVQAGESLKLSCKAS (SEQ ID NO: 16) or a variant thereof comprising up to 3 amino acid substitutions, the FR2 comprises an amino acid sequence of WHRQRAGQQQRELV (SEQ ID NO: 21) or a variant thereof comprising up to 3 amino acid substitutions, the FR3 comprises an amino acid sequence of YADSVKGRFTLSSDSANDVVYLEMTDLKPEDTAVYYCH (SEQ ID NO: 26) or a variant thereof comprising up to 3 amino acid substitutions, and the FR4 comprises an amino acid sequence of GPGTQVTVSS (SEQ ID NO: 31) or a variant thereof comprising up to 3 amino acid substitutions;
the CDR1 comprises an amino acid sequence of GFTLSTLDMR (SEQ ID NO: 2), the CDR2 comprises an amino acid sequence of STVSPDGNTY (SEQ ID NO: 7), the CDR3 comprises an amino acid sequence of ADISIAAHEGGLPYW (SEQ ID NO: 12), the FR1 comprises an amino acid sequence of QVQLVQSGGGLVQPGGSLRLSCAAS (SEQ ID NO: 17) or a variant thereof comprising up to 3 amino acid substitutions, the FR2 comprises an amino acid sequence of WFRQAPGKGFEWV (SEQ ID NO: 22) or a variant thereof comprising up to 3 amino acid substitutions, the FR3 comprises an amino acid sequence of YSDSAKGRFAISRDVAKNTVNLQMSSLKPEDTAVYYCN (SEQ ID NO: 27) or a variant thereof comprising up to 3 amino acid substitutions, and the FR4 comprises an amino acid sequence of GKGTPVTVSS (SEQ ID NO: 32) or a variant thereof comprising up to 3 amino acid substitutions;
the CDR1 comprises an amino acid sequence of GFDFSRYDMG (SEQ ID NO: 3), the CDR2 comprises an amino acid sequence of SGIKSGGGPTV (SEQ ID NO: 8), the CDR3 comprises an amino acid sequence of DPDDHSGSYWGDYW (SEQ ID NO: 13), the FR1 comprises an amino acid sequence of DVQLVESGGGMVQPGGSLRLSCVTS (SEQ ID NO: 18) or a variant thereof comprising up to 3 amino acid substitutions, the FR2 comprises an amino acid sequence of WVRQAPGKGPEWV (SEQ ID NO: 23) or a variant thereof comprising up to 3 amino acid substitutions, the FR3 comprises an amino acid sequence of YLDSVKGRFTVSRDNAKNTLYLQMNNLKPEDTARYYCA (SEQ ID NO: 28) or a variant thereof comprising up to 3 amino acid substitutions, and the FR4 comprises an amino acid sequence of GQGTLVTVSS (SEQ ID NO: 33) or a variant thereof comprising up to 3 amino acid substitutions;
the CDR1 comprises an amino acid sequence of GSX¹X²RVNAMG (SEQ ID NO: 4) wherein X¹ is T or D and X² is S or Y, the CDR2 comprises an amino acid sequence of AAIDRSGATV (SEQ ID NO: 9), the CDR3 comprises an amino acid sequence of SGVLGSW (SEQ ID NO: 14), the FR1 comprises an amino acid sequence of QVQLVESGGGLAQAGGSLQLSCAAS (SEQ ID NO: 19) or a variant thereof comprising up to 3 amino acid substitutions, the FR2 comprises an amino acid sequence of WYRQTPGKERELV (SEQ ID NO: 24) or a variant thereof comprising up to 3 amino acid substitutions, the FR3 comprises an amino acid sequence of YSESVRGRFTISKNDX³KX⁴IVWLQMNNLTTEDTAVYYCR (SEQ ID NO: 29) wherein X³ is A, Y, or W and X⁴ is N, D, H, or Y or a variant thereof comprising up to 3 amino acid substitutions, and the FR4 comprises an amino acid sequence of GLGTQVTVSS (SEQ ID NO: 34) or a variant thereof comprising up to 3 amino acid substitutions; or
the CDR1 comprises an amino acid sequence of GNTFSINSMA (SEQ ID NO: 5), the CDR2 comprises an amino acid sequence of AELHKTDRPGSVAAN (SEQ ID NO: 10), the CDR3 comprises an amino acid sequence of ATGLWRDHEVW (SEQ ID NO: 15), the FR1 comprises an amino acid sequence of QVQLVESGGGLVQAGESLNLSCTAS (SEQ ID NO: 20) or a variant thereof comprising up to 3 amino acid substitutions, the FR2 comprises an amino acid sequence of WYRQAPGKQRELV (SEQ ID NO: 25) or a variant thereof comprising up to 3 amino acid substitutions, the FR3 comprises an amino acid sequence of YADSVKGRFTIATDNSRDTMYLQMTNLKSADTATYFCY (SEQ ID NO: 30) or a variant thereof comprising up to 3 amino acid substitutions, and the FR4 comprises an amino acid sequence of GQGTLVTVSS (SEQ ID NO: 35) or a variant thereof comprising up to 3 amino acid substitutions.

12. The isolated anti-opioid construct of claim 11, wherein the sdAb moiety comprises an amino acid sequence having at least 95% sequence identity to an sdAb sequence selected from the group consisting of SEQ ID NOs:36-44.

13. The isolated anti-opioid construct of claim 1, wherein the sdAb moiety comprises an FR1, an FR2, an FR3, and an FR4, wherein the CDR1 comprises an amino acid sequence of GSX¹X²RVNAMG (SEQ ID NO: 4) wherein X¹ is T or D and X² is S or Y, the CDR2 comprises an amino acid sequence of AAIDRSGATV (SEQ ID NO: 9), the CDR3 comprises an amino acid sequence of SGVLGSW (SEQ ID NO: 14), the FR1 comprises an amino acid sequence of QVQLVESGGGLAQAGGSLQLSCAAS (SEQ ID NO: 19) or a variant thereof comprising up to 3 amino acid substitutions, the FR2 comprises an amino acid sequence of WYRQTPGKERELV (SEQ ID NO: 24) or a variant thereof comprising up to 3 amino acid substitutions, the FR3 comprises an amino acid sequence of YSESVRGRFTISKNDX³KX⁴IVWLQMNNLTTEDTAVYYCR (SEQ ID NO: 29) wherein X³ is A, Y, or W and X⁴ is D or a variant thereof comprising up to 3 amino acid substitutions with the proviso that the D at X⁴ is not substituted, and the FR4 comprises an amino acid sequence of GLGTQVTVSS (SEQ ID NO: 34) or a variant thereof comprising up to 3 amino acid substitutions.

14. The isolated anti-opioid construct of claim 1, wherein the sdAb moiety comprises an amino acid sequence having at least 95% sequence identity to an sdAb sequence selected from the group consisting of SEQ ID NOs:36-44.

15. The isolated anti-opioid construct of claim 1, wherein the sdAb moiety:
comprises an amino acid sequence having at least 95% sequence identity to an sdAb sequence selected from the group consisting of SEQ ID NOs:39 and 41-44; and
comprises any one or more, any two or more, any three or more, any four or more, any five or more, any six or more, any seven or more, any eight or more, any nine or more, or each of:
an alanine at a position corresponding to position 24 in the sdAb sequence;
a serine or tyrosine at a position corresponding to position 29 in the sdAb sequence;
a valine at a position corresponding to position 31 in the sdAb sequence;
an asparagine at a position corresponding to position 32 in the sdAb sequence;
a methionine at a position corresponding to position 34 in the sdAb sequence;
an arginine at a position corresponding to position 53 in the sdAb sequence;
a lysine at a position corresponding to position 71 in the sdAb sequence;
an alanine, tyrosine, or tryptophan at a position corresponding to position 74 in the sdAb sequence;
an asparagine, aspartate, or histidine at a position corresponding to position 76 in the sdAb sequence; and
a valine at a position corresponding to position 78 in the sdAb sequence.

16. The isolated anti-opioid construct of claim 1, wherein the isolated anti-opioid construct comprises the sdAb moiety fused to a second antibody moiety.

17. The isolated anti-opioid construct of claim 16, wherein the sdAb moiety is fused to the second antibody moiety via a linker.

18. The isolated anti-opioid construct of claim 16, wherein the second antibody moiety is a second copy of the sdAb moiety.

19. A pharmaceutical composition comprising the isolated anti-opioid construct of claim 1 or a nucleic acid configured to express the isolated anti-opioid construct.

20. A method comprising administering the pharmaceutical composition of claim 19 to an individual to thereby bind the anti-opioid construct to an opioid present within a body of the individual, wherein the opioid is selected from the group consisting of fentanyl and carfentanil.
